(19) United States
(12) Patent Application Publication
(10) Pub. No.: US 2024/0059669 A1
(43) Pub. Date: Feb. 22, 2024

(54) COCRYSTAL OF A CDK INHIBITOR

(71) Applicant: AURIGENE ONCOLOGY LIMITED, Bangalore (IN)

(72) Inventors: Uday Bhat, Bangalore (IN); Ranadeep Bokalial, Bengaluru (IN); Sangamesh Eshwarappa Badiger, Bangalore (IN); Krishnaswamy Devanathan, Bangalore (IN)

(21) Appl. No.: 18/257,962

(22) PCT Filed: Dec. 17, 2021
(86) PCT No.: PCT/IB2021/061895

§ 371 Date: Jun. 16, 2023

(30) Foreign Application Priority Data
Dec. 18, 2020 (IN) 202041055174

(51) Int. Cl. C07D 401/12

(52) U.S. Cl. CPC C07D 401/12; C07B 2200/13

(57) ABSTRACT

The present invention relates to compound of formula (I) fumarate and its crystalline form and methods of their preparation.

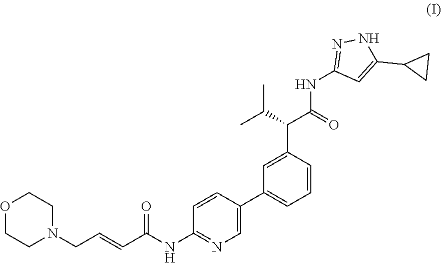 [SMILES: CC(C)[C@H](C(=O)Nc1cc(C2CC2)[nH]n1)c1cccc(-c2ccc(NC(=O)/C=C/CN3CCOCC3)nc2)c1]

The invention also relates to preparations suitable for pharmaceutical uses for treatment of various diseases or disorders mediated by CDK7, particularly cancer or other proliferative diseases.

### CROSS-REFERENCE TO RELATED APPLICATION

This application claims the benefit of Indian provisional application number 202041055174, filed on 18 Dec. 2020, the specification of which is hereby incorporated by reference in its entirety.

### FIELD OF THE INVENTION

The present invention is directed to compound of formula (I) fumarate including its crystalline form and methods of their preparation. The present invention also relates to pharmaceutical compositions comprising the said compound of formula (I) fumarate, crystalline form thereof and methods of their use as therapeutic agent.

### BACKGROUND OF THE INVENTION

CDK7, which complexes with cyclin H and MAT1, phosphorylates the cell cycle CDKs in the activation of T-loop, to promote their activities (Fisher et al., Cell., August 26;78(4):713-24, 1994). As such, it has been proposed that inhibiting CDK7 would provide a potent means of inhibiting cell cycle progression, which may be especially relevant given that there is compelling evidence from gene knockout studies in mice for lack of an absolute requirement for CDK2, CDK4 and CDK6 for the cell cycle, at least in most cell types (Malumbres et al., Nature Cell Biology, 11, 1275-1276, 2009), whilst different tumors appear to require some, but be independent of other interphase CDKs (CDK2, CDK4, CDK6). Recent genetic and biochemical studies have confirmed the importance of CDK7 for cell cycle progression (Larochelle et al., Mol Cell., March 23;25(6):839-50. 2007; Ganuza et al., EMBO J., May 30; 31(11): 2498-510, 2012).

Cyclin-dependent kinase 7 (CDK7) activates cell cycle CDKs and is a member of the general Transcription factor II Human (TFIIH). CDK7 also plays a role in transcription and possibly in DNA repair. The trimeric Cak complex CDK7/CyclinH/MAT1 is also a component of TFIIH, the general transcription/DNA repair factor IIH (Morgan, D. O., Annu. Rev. Cell Dev. Biol. 13, 261-91, 1997). As a TFIIH subunit, CDK7 phosphorylates the CTD (Carboxy-Terminal-Domain) of the largest subunit of RNA polymerase II (pol II). The CTD of mammalian pol II consists of 52 heptad repeats with the consensus sequence ¹YSPTSPS⁷ and the phosphorylation status of the Ser residues at positions 2 and 5 has been shown to be important in the activation of RNAP-II indicating that it is likely to have a crucial role in the function of the CTD. CDK7, which primarily phosphorylates Ser-5 (PSS) of RNAP-II at the promoter as part of transcriptional initiation (Gomes et al., Genes Dev. 2006 March 1; 20(5):601-12, 2006), in contrast with CDK9, which phosphorylates both Ser-2 and Ser-5 of the CTD heptad (Pinhero et al., Eur. J. Biochem., 271, pp. 1004-1014, 2004).

In addition to CDK7, other CDKs have been reported to phosphorylate and regulate RNA pol (II) CTD. The other CDKs include, Cdk9/Cyclin T1 or T2 that constitute the active form of the positive transcription elongation factor (P-TEFb) (Peterlin and Price, Mol Cell., August 4; 23(3): 297-305,2006) and Cdkl2/Cyclin K and Cdkl3/Cyclin K as the latest members of RNAPII CTD kinases (Bartkowiak et al., Genes Dev., October 1 5;24(20):2303-16, 2010; Blazek et al., Genes Dev. October 15;25(20):2158-72, 2011).

Disruption of RNAP II CTD phosphorylation has been shown to preferentially effect proteins with short half-lives, including those of the anti-apoptotic BCL-2 family. (Konig et al., Blood, 1, 4307-4312, 1997; The transcriptional non-selective cyclin-dependent kinase inhibitor flavopiridol induces apoptosis in multiple myeloma cells through transcriptional repression and down-regulation of Mcl-1; (Gojo et al., Clin. Cancer Res. 8, 3527-3538, 2002).

This suggests that the CDK7 enzyme complexes are involved in multiple functions in the cell: cell cycle control, transcription regulation and DNA repair. It is surprising to find one kinase involved in such diverse cellular processes, some of which are even mutually exclusive. It also is puzzling that multiple attempts to find cell cycle dependent changes in CDK7 kinase activity remained unsuccessful. This is unexpected since activity and phosphorylation state of its substrate, CDC2, fluctuate during the cell cycle. In fact, it is shown that cdk7 activity is required for the activation of both Cdc2/Cyclin A and Cdc2/Cyclin B complexes, and for cell division. (Larochelle, S. et al. Genes Dev 12,370-81, 1998). Indeed, flavopiridol, a non-selective pan-CDK inhibitor that targets CTD kinases, has demonstrated efficacy for the treatment of chronic lymphocytic leukemia (CLL), but suffers from a poor toxicity profile (Lin et al.,]. Clin. Oncol. 27, 6012-6018, 2009; Christian et al., Clin. Lymphoma Myeloma,9, Suppl. 3, 5179-S185, 2009).

International publication, WO2016193939 describes CDK7 inhibitors, processes for the preparation thereof, pharmaceutical compositions comprising them and their uses as therapeutic agents as protein kinase inhibitors in the treatment of various disorders, particularly in the treatment of cancer and other proliferative disorders, which is incorporated herein by reference for all purposes. Inhibitors of CDK7 are currently being developed for the treatment of cancer. For drug development, it is typically advantageous to employ a form of the drug having desirable properties with respect to its preparation, purification, reproducibility, stability, bioavailability, and other characteristics.

Accordingly, there is a need for new forms, salts and/or cocrystals of CDK7-inhibiting molecules for preparing pharmaceutically useful formulations and dosage forms with suitable properties related to, for example, facilitating the manufacture of safe, effective, and high-quality drug products.

### SUMMARY OF THE INVENTION

Compound of formula (I) is also known as (S, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide.

The present disclosure provides processes and synthesis of the compound of formula (I) with high yield and purity. The compound of formula (I) fumarate, as invented, exhibits unexpected physicochemical properties, for example, greater stability, greater purity and lesser hygroscopicity compared to its free base form.

In an aspect, the present invention provides compound of formula (I) fumarate;

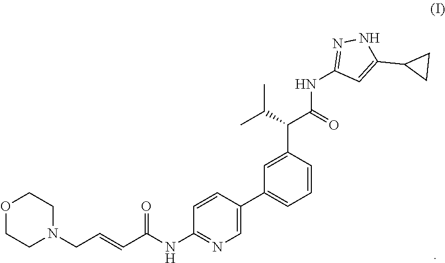 [SMILES: CC(C)[C@H](C(=O)Nc1cc(C2CC2)[nH]n1)c1cccc(-c2ccc(NC(=O)/C=C/CN3CCOCC3)nc2)c1]

In an aspect, the present invention provides cocrystal comprising compound of formula (I) and fumaric acid.

In another aspect, the present invention provides compound of formula (I)-fumaric acid (1:1) cocrystal:

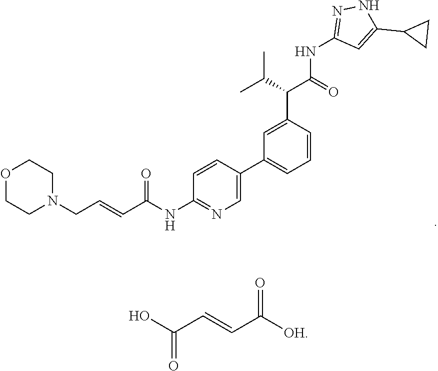 [SMILES: CC(C)[C@H](C(=O)Nc1cc(C2CC2)[nH]n1)c1cccc(-c2ccc(NC(=O)/C=C/CN3CCOCC3)nc2)c1 ; O=C(O)/C=C/C(=O)O]

In further aspect, the present invention relates to methods of preparation of compound of formula (I) fumarate.

In further aspect, the present invention relates to methods of preparation of crystalline compound of formula (I) fumarate.

In further aspect, the present invention provides a pharmaceutical composition comprising compound of formula (I) fumarate.

In another aspect, the present invention provides uses of compound of formula (I) fumarate for the treatment of a variety of diseases, disorders, or conditions as described herein.

### BRIEF DESCRIPTION OF THE DRAWINGS

FIG. 1 shows an asymmetric unit of single crystal of compound of formula (I) fumarate.

FIG. 2 shows an XRPD pattern of compound of formula (I) fumarate Form 1 collected at 25° C.

FIG. 3 shows a differential scanning calorimetry (DSC) thermogram of compound of formula (I) fumarate.

FIG. 4 shows a thermogravimetric analysis (TGA) of compound of formula (I) fumarate.

FIG. 5A shows an optical and polarized light microscopy study of compound of formula (I) fumarate

FIG. 5B shows a polarized light microscopy study of compound of formula (I) fumarate

FIG. 6A shows the overlay of VT-XRD patterns of compound of formula (I) fumarate Form 1 collected at 25° C., 40° C. and 60° C.

FIG. 6B shows the overlay of VH-XRPD patterns of compound of formula (I) fumarate Form 1 collected at various humidity conditions.

FIG. 7 shows the overlay of XRPD pattern of compound of formula (I) fumarate Form 1 exposed to different conditions.

FIG. 8 shows a dynamic vapor sorption (DVS) of compound of formula (I) fumarate Form 1.

### DETAILED DESCRIPTION OF THE INVENTION

As used in the present specification, the following words and phrases are generally intended to have the meanings as set forth below, except to the extent that the context in which they are used indicates otherwise.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans.

As used herein, the term “treat”, “treating” or “treatment” refers to one or more of (1) inhibiting the disease; for example, inhibiting a disease, condition, syndrome or disorder in an individual who is experiencing or displaying the pathology or symptomatology of the disease, condition or disorder (i.e., arresting further development of the pathology and/or symptomatology); and (2) ameliorating the disease; for example, ameliorating or abrogating a disease, condition or disorder in an individual who is experiencing or displaying the pathology or symptomatology of the disease, condition or disorder (i.e., reversing the pathology and/or symptomatology) such as decreasing the severity of disease. In one embodiment, the term “treat”, “treating” or “treatment” refers to preventing or delaying the onset or development or progression of the disease, condition, syndrome or disorder.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds. ; Gower Publishing Company: 2007; Pharmaceutical Preformulation and Formulation, 2nd ed. ; Gibson Ed.; CRC Press LLC: Boca Raton, Fla., 2009.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols or acetonitrile (ACN) are preferred.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and orppharyngeal cancer; Lung: bronchogenic carcinoma (squamous cell, undifferentiated small cell, undifferentiated large cell, adenocarcinoma, non-small cell lung cancer), alveolar (bronchiolar) carcinoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, papillary carcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

As used herein, “cocrystal(s)” refers to single phase crystalline materials comprising at least two non-volatile components in the same crystal lattice in a specific stoichiometric (molar) ratio, where the arrangement in the crystal lattice is not based on ionic bonds (as with salts) or covalent interactions and at least two of the components are solids at room temperature. Cocrystals consist of two or more components that form a unique crystalline structure having unique properties. Cocrystals can encompass hydrates, solvates and clathrates.

As used herein, “crystalline” or “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content.

As used herein, “therapeutically effective amount” refers to an amount that is sufficient to effect treatment, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

The term “substantially pure”, as used herein, refers to a crystalline polymorph that is greater than 90% pure, meaning that it contains less than 10% of any other compound, or an alternative polymorph of the crystalline form. Preferably, the crystalline polymorph is greater than 95% pure, or even greater than 98% pure.

The phrase “substantially as shown in FIG.” refers to a pattern with at least 50%, or at least 60%, or at least 70%, or at least 80%, or at least 90%, or at least 95% or at least 99% of its value appears in FIG.

As used herein, the term “about” when referring to a number or a numerical range means that the number or numerical range referred to, is an approximation within experimental variability (or within statistical experimental error), and thus the number or numerical range may vary from, for example, between 1% and 15% of the stated number or numerical range.

Each embodiment is provided by way of explanation of the invention, and not by way of limitation of the invention. In fact, it will be apparent to those skilled in the art that various modifications and variations can be made to the compounds, compositions, and methods described herein without departing from the scope or spirit of the invention. For instance, features illustrated or described as part of one embodiment can be applied to another embodiment to yield a still further embodiment. Thus, it is intended that the present invention include such modifications and variations and their equivalents. Other objects, features, and aspects of the present invention are disclosed in, or are obvious from, the following detailed description. It is to be understood by one of ordinary skill in the art that the present discussion is a description of exemplary embodiments only and is not to be construed as limiting the broader aspects of the present invention.

### Compound of Formula (I) Fumarate

In one embodiment, the present invention provides a compound of formula (I) fumarate;

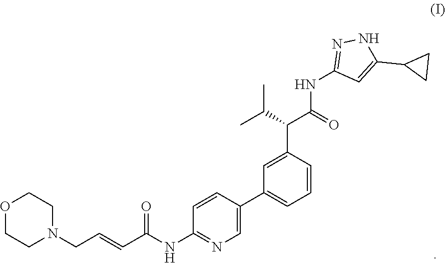 [SMILES: CC(C)[C@H](C(=O)Nc1cc(C2CC2)[nH]n1)c1cccc(-c2ccc(NC(=O)/C=C/CN3CCOCC3)nc2)c1]

In one embodiment, the present invention provides compound of formula (I)-fumaric acid cocrystal.

In one embodiment, the molar ratio of compound of formula (I) to fumaric acid is 1:1.

In one embodiment, the present invention provides a crystalline compound of formula (I) fumarate.

In one embodiment, the present invention provides a crystalline compound of formula (I) fumarate having Form 1.

### Identification Analysis of Compound of Formula (I) Fumarate

The different crystalline forms can be identified by solid state characterization methods such as by X-ray powder diffraction (XRPD). Other characterization methods such as differential scanning calorimetry (DSC), thermogravimetric analysis (TGA), dynamic vapor sorption (DVS), and the like, further help identify the form as well as determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed, or existing peaks may disappear, depending on the type of the instrument or the settings. As used herein, the term “peak” refers to a reflection having a relative height intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In one embodiment, the polymorph of the crystalline compound is characterized by powder X-ray diffraction (XRD). θ represents the diffraction angle which is measured in degrees. In one embodiment, the diffractometer used in XRD measures the diffraction angle as two times the diffraction angle theta (θ). Thus, in one embodiment, the diffraction patterns described herein refer to X-ray intensity measured against angle 2-theta (θ).

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

In one embodiment, the compound of formula (I) fumarate is in crystalline form. In one embodiment, the compound of formula (I) fumarate is substantially crystalline. In one embodiment, “substantially crystalline” refers to the compound of formula (I) fumarate that is at least a particular weight percent crystalline. Particular weight percentages include 50%, 60%, 70%, 75%, 80%, 85%, 87%, 88%, 89%, 90%, 91%, 92%, 93%, 94%, 95%, 96%, 97%, 98%, 99%, 99.5% and 99.9%. In one embodiment, substantially crystalline refers to the compound of formula (I) fumarate that is at least 70% crystalline. In one embodiment, substantially crystalline refers to the compound of formula (I) fumarate that is at least 80% crystalline. In one embodiment, substantially crystalline refers to the compound of formula (I) fumarate that is at least 85% crystalline. In one embodiment, substantially crystalline refers to the compound of formula (I) fumarate that is at least at least 90% crystalline. In some embodiments, substantially crystalline refers to the compound of formula (I) fumarate that is at least 95% crystalline. In one embodiment, the compound of formula (I) fumarate is 99.9% crystalline.

In one embodiment, the compound of formula (I) fumarate is in crystalline Form 1.

In one embodiment, the crystalline compound of formula (I) fumarate is anhydrous.

In one embodiment, the present invention provides a cocrystal of compound of formula (I) fumarate.

In one embodiment, the crystalline compound of formula (I) fumarate is characterized by X-ray diffraction analysis.

In one embodiment, the compound of formula (I)-fumarate cocrystal is characterized by X-ray powder diffraction pattern comprising at least one peak at 2θ angles about 5.0±0.2.

In one embodiment, the compound of formula (I)-fumarate cocrystal is characterized by X-ray powder diffraction pattern comprising at least one peak at 2θ angles about 10.0±0.2.

In one embodiment, the compound of formula (I)-fumarate cocrystal is characterized by X-ray powder diffraction pattern comprising at least one peak at 2θ angles about 15.0±0.2.

In one embodiment, the compound of formula (I)-fumarate cocrystal is characterized by X-ray powder diffraction pattern comprising at least one peak at 2θ angles about 12.0±0.2.

In one embodiment, the compound of formula (I)-fumarate cocrystal is characterized by X-ray powder diffraction pattern comprising at least one peak at 2θ angles about 18.0±0.2.

In one embodiment, the compound of formula (I)-fumarate cocrystal is characterized by X-ray powder diffraction pattern comprising at least one peak at 2θ angles about 22.0±0.2.

In one embodiment, the compound of formula (I)-fumarate cocrystal is characterized by X-ray powder diffraction pattern comprising at least one peak at 2θ angles about 24.0±0.2.

In one embodiment, the compound of formula (I)-fumaric acid cocrystal has 20 values characterized by X-ray powder diffraction pattern comprising peaks at about: 5.0±0.2, 10.0±0.2, 10.5±0.2, 15.0±0.2, 18.7±0.2, and 19.8±0.2.

In one embodiment, the compound of formula (I)-fumaric acid cocrystal has 20 values characterized by X-ray powder diffraction pattern comprising at least one, two or three peak(s) at about: 5.0±0.2, 10.0±0.2, 10.5±0.2, 15.0±0.2, 18.7±0.2, and 19.8±0.2.

In one embodiment, the compound of formula (I)-fumaric acid cocrystal has 20 values characterized by X-ray powder diffraction pattern comprising peaks at about: 5.0±0.2, 10.0±0.2, 10.5±0.2, 15.0±0.2, 18.7±0.2, 19.8±0.2, 20.0±0.2, 22.0±0.2 and 22.5±0.2.

In one embodiment, the compound of formula (I)-fumaric acid cocrystal has 20 values characterized by X-ray powder diffraction pattern comprising at least four peaks at about: 5.0±0.2, 10.0±0.2, 10.5±0.2, 15.0±0.2, 18.7±0.2, 19.8±0.2, 20.0±0.2, 22.0±0.2 and 22.5±0.2.

In one embodiment, the compound of formula (I)-fumaric acid cocrystal has 20 values 5.0±0.2, 10.0±0.2 10.5±0.2, 12.0±0.2, 13.6±0.2, 14.8±0.2, 15.0±0.2, 17.6±0.2, 18.7±0.2, 19.8±0.2, 20.0±0.2, 21.2±0.2, 22.0±0.2, 22.5±0.2, 24.0±0.2, 25.0±0.2, 26.1±0.2, and 27.4±0.2.

In one embodiment, a compound of formula (I)-fumaric acid cocrystal has 20 values characterized by X-ray powder diffraction pattern comprising peaks at about: 5.0±0.2, 10.0±0.2 10.5±0.2, 12.0±0.2, 14.8±0.2, 15.0±0.2, 15.6±0.2, 17.6±0.2, 18.7±0.2, 19.8±0.2, 20.0±0.2, 20.1±0.2, 21.2±0.2, 22.0±0.2, 22.5±0.2, 23.4±0.2, 24.0±0.2, 25.0±0.2, 26.1±0.2, 26.8±0.2, 27.4±0.2, and 36.6±0.2.

In one embodiment, the compound of formula (I)-fumaric acid cocrystal has 20 values characterized by X-ray powder diffraction pattern comprising peaks at about: 5.0±0.2, 10.0±0.2 10.5±0.2, 12.0±0.2, 13.6±0.2, 14.8±0.2, 15.0±0.2, 15.6±0.2, 16.5±0.2, 17.6±0.2, 18.7±0.2, 19.3±0.2, 19.8±0.2, 20.0±0.2, 20.1±0.2, 21.2±0.2, 22.0±0.2, 22.5±0.2, 23.0±0.2, 23.4±0.2, 24.0±0.2, 25.0±0.2, 25.6±0.2, 26.1±0.2, 26.8±0.2, 27.4±0.2, 28.9±0.2, 29.3±0.2, 30.1±0.2, 30.5±0.2, 31.4±0.2, 32.2±0.2, 33.4±0.2, 36.1±0.2, 36.6±0.2, 38.0±0.2, 39.2±0.2, 40.2±0.2, 40.6±0.2, and 41.0±0.2.

TABLE 1
Exemplary peaks of Form 1

| Index | Pos. [°2 Theta] | d-spacing [Å] | Rel. Int. [%] |
|---|---|---|---|
| 1 | 5.011 | 17.62253 | 100.0 |
| 2 | 9.992 | 8.84506 | 8.2 |
| 3 | 10.535 | 8.39092 | 6.3 |
| 4 | 11.953 | 7.39805 | 4.5 |
| 5 | 13.617 | 6.49743 | 1.3 |
| 6 | 14.748 | 6.00196 | 4.5 |
| 7 | 14.994 | 5.90370 | 42.0 |
| 8 | 15.557 | 5.69151 | 1.2 |
| 9 | 16.533 | 5.35770 | 0.6 |
| 10 | 17.673 | 5.01457 | 2.7 |
| 11 | 18.741 | 4.73116 | 7.3 |
| 12 | 19.306 | 4.59393 | 0.4 |
| 13 | 19.865 | 4.46590 | 6.3 |
| 14 | 20.000 | 4.43594 | 5.3 |
| 15 | 20.094 | 4.41548 | 1.8 |
| 16 | 21.194 | 4.18871 | 3.8 |
| 17 | 22.090 | 4.02072 | 5.7 |
| 18 | 22.509 | 3.94690 | 5.6 |
| 19 | 23.026 | 3.85933 | 0.7 |
| 20 | 23.420 | 3.79539 | 1.7 |
| 21 | 24.034 | 3.69975 | 3.7 |
| 22 | 25.005 | 3.55820 | 3.1 |
| 23 | 25.628 | 3.47313 | 0.6 |
| 24 | 26.077 | 3.41440 | 3.1 |
| 25 | 26.812 | 3.32242 | 1.0 |
| 26 | 27.412 | 3.25102 | 2.6 |
| 27 | 28.932 | 3.08358 | 0.8 |
| 28 | 29.250 | 3.05084 | 0.7 |
| 29 | 30.116 | 2.96499 | 0.6 |
| 30 | 30.542 | 2.92463 | 0.3 |
| 31 | 31.360 | 2.85014 | 0.4 |
| 32 | 32.235 | 2.77479 | 0.3 |
| 33 | 33.418 | 2.67917 | 0.5 |
| 34 | 36.070 | 2.48804 | 0.6 |
| 35 | 36.634 | 2.45103 | 1.2 |
| 36 | 37.951 | 2.36896 | 0.8 |
| 37 | 39.161 | 2.29852 | 0.4 |
| 38 | 40.245 | 2.23904 | 0.5 |
| 39 | 40.590 | 2.22082 | 0.7 |
| 40 | 40.953 | 2.20195 | 0.4 |

Provided herein is characterizing information to describe any solid forms of compound of formula (I) fumarate. It should be understood, however, that not all such information is required for one skilled in the art to determine that such particular form is present in a given composition, but that the determination of a particular form can be achieved using any portion of the characterizing information that one skilled in the art would recognize as sufficient for establishing the presence of a particular form, e.g., even a distinguishing peak can be sufficient for one skilled in the art to appreciate that such particular form is present.

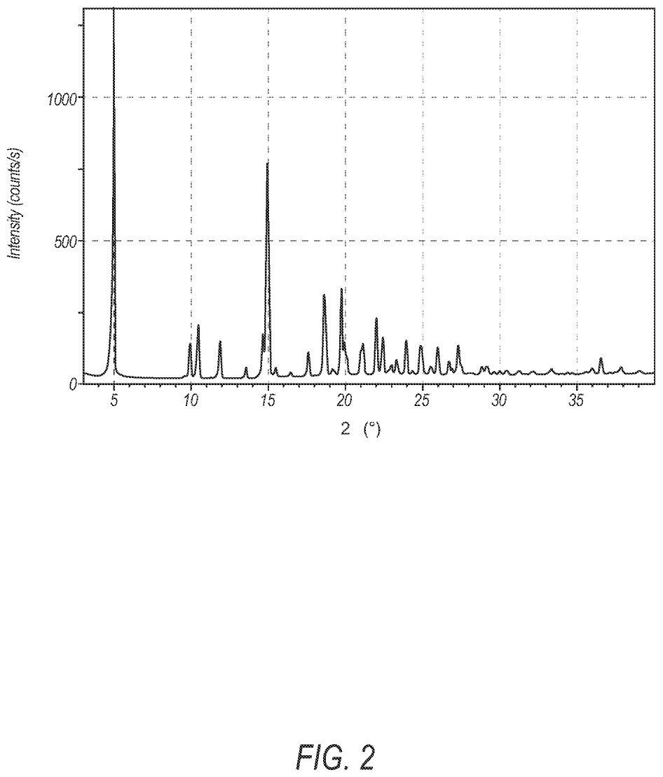

In one embodiment, the compound of formula (I)-fumaric acid cocrystal has an XPRD pattern substantially as shown in FIG. 2.

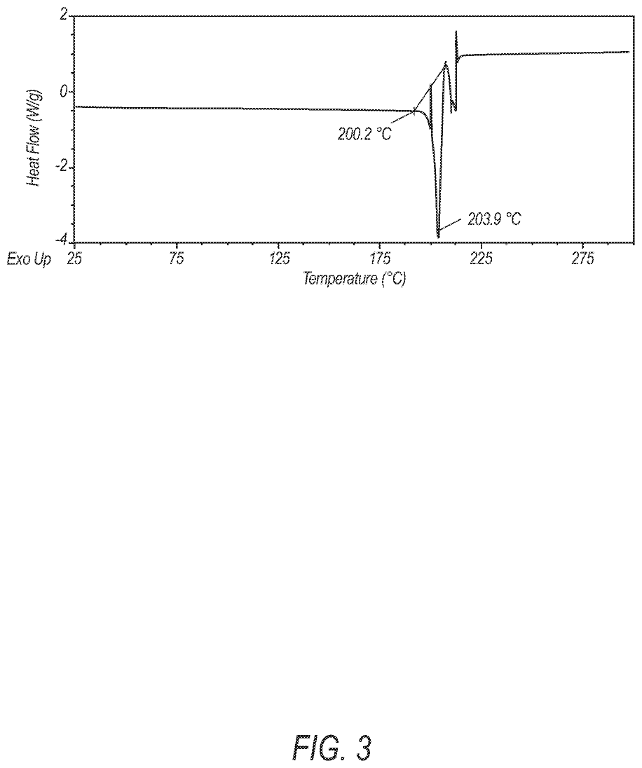

In one embodiment, the compound of formula (I)-fumaric acid cocrystal by differential scanning calorimetry (DSC) shows an endotherm with a peak temperature from about 190° C. to about 210° C. corresponding to the melting. In some embodiments, the compound of formula (I)-fumaric acid cocrystal shows an endotherm with a peak temperature in the range selected from about 195° C. to about 210° C. corresponding to the melting. In some embodiments, the compound of formula (I)-fumaric acid cocrystal shows an endotherm with a peak temperature in the range selected from about 195° C. to about 205° C., about 198° C. to about 205° C. and about 199° C. to about 204° C. by DSC corresponding to the melting. In one embodiment, the compound of formula (I)-fumaric acid cocrystal shows an endotherm with a peak temperature at 203° C.±3° C. See, FIG. 3.

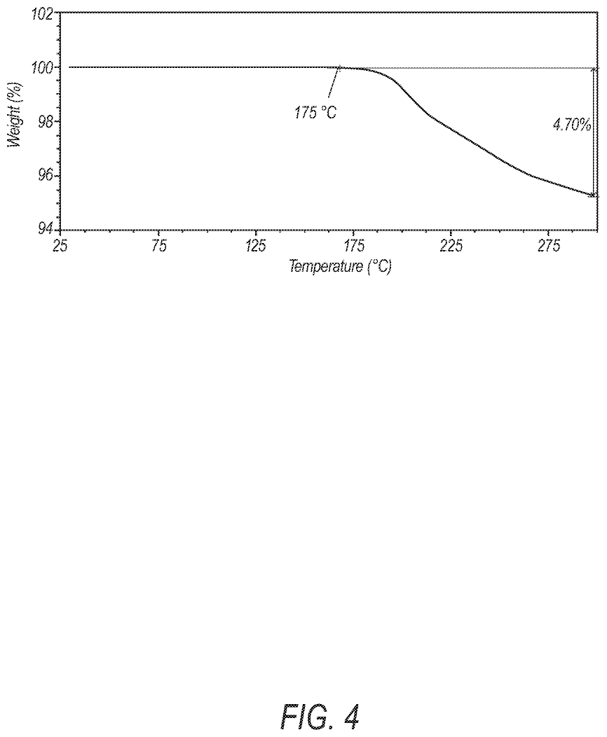

In one embodiment, compound of formula (I)-fumaric acid cocrystal is having a thermogravimetric analysis substantially as shown in FIG. 4.

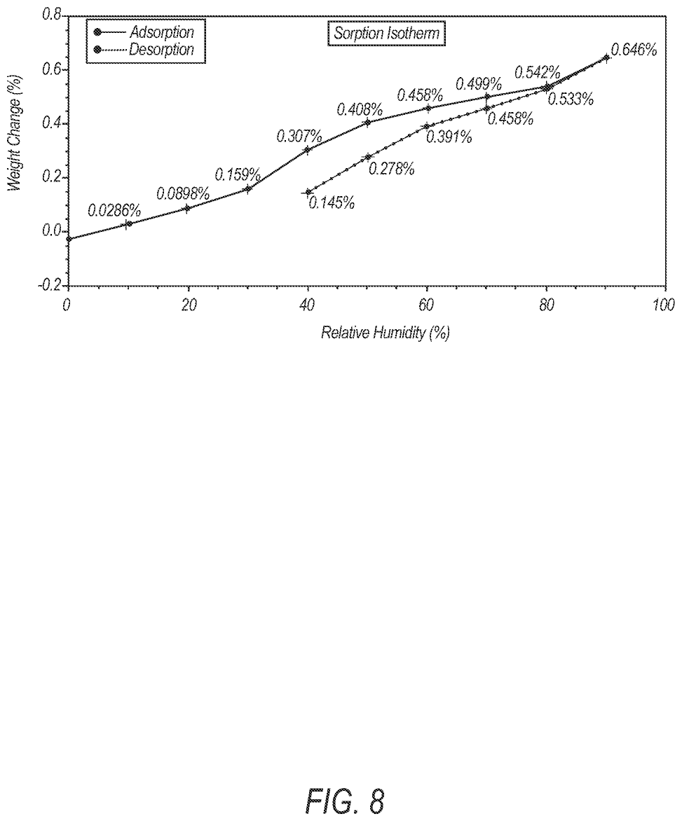

In one embodiment, compound of formula (I)-fumaric acid cocrystal is having a dynamic vapor sorption substantially as shown in FIG. 8.

### Methods of Preparing Compound of Formula (I) Fumarate

In one embodiment, the present invention provides a method for preparing a compound of formula (I) fumarate comprising:
a) adding fumaric acid to a mixture comprising the compound of formula (I)

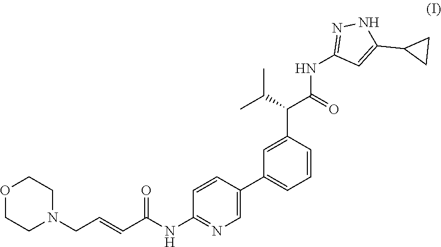 [SMILES: CC(C)[C@H](C(=O)Nc1cc(C2CC2)[nH]n1)c1cccc(-c2ccc(NC(=O)/C=C/CN3CCOCC3)nc2)c1]

and a solvent; and
b) obtaining the compound of formula (I) fumarate from the mixture.

In one embodiment, the mixture comprises a solution of the compound of formula (I). In one embodiment, the solution comprises a solid crude material comprising the compound of formula (I) dissolved in a solvent. In one embodiment, a solid crude material comprises about 70% to about 90% compound of formula (I).

In one embodiment, in step a) of the preparation of compound of formula (I) fumarate, the fumaric acid is dissolved in a solvent.

In one embodiment, in the preparation of compound of formula (I) fumarate, the solvent is methanol, acetonitrile, acetone, anisole, dichloromethane, dichloroethane, ethanol, methyl acetate, n-propyl acetate, isopropyl alcohol, isopropyl acetate, propanol, butanol, pentanol, n-butyl acetate, isobutyl acetate, isobutylene acetate, methylcyclohexane, methyl tert-butyl ether, n-hexane, n-heptane, tetrahydrofuran, or water, or any mixtures thereof.

In one embodiment, in the preparation of compound of formula (I) fumarate, the solvent is methanol, acetone, dichloroethane, ethanol, methyl acetate, n-propyl acetate, isopropyl alcohol, isopropyl acetate, propanol, butanol, n-butyl acetate, methylcyclohexane, methyl tert-butyl ether, n-hexane, n-heptane, tetrahydrofuran, or water, or any mixtures thereof.

In one embodiment, in the preparation of compound of formula (I) fumarate, the solvent is methanol, ethanol, methyl acetate, isopropyl alcohol, isopropyl acetate, propanol, butanol, n-butyl acetate, methylcyclohexane, or water, or any mixtures thereof

In one embodiment, in the preparation of compound of formula (I) fumarate, the solvent is methanol, ethanol, methyl acetate, isopropyl alcohol, isopropyl acetate, propanol, n-hexane, n-heptane or water, or any mixtures thereof.

In one embodiment, in the preparation of compound of formula (I) fumarate, the solvent is methanol, ethanol, isopropyl alcohol, isopropyl acetate or water, or any mixtures thereof

In one embodiment, in the preparation of compound of formula (I) fumarate, the solvent is methanol or ethanol.

In one embodiment, in the preparation of compound of formula (I) fumarate, the solvent is methanol.

In one embodiment, in the preparation of compound of formula (I) fumarate, obtaining the compound of formula (I) fumarate comprises:
i. stirring the mixture comprising the compound of formula (I) and fumaric acid;
ii. cooling the mixture to ambient temperature thereby forming the suspension;
iii. isolating the compound of formula (I) fumarate from the suspension.

In one embodiment, the mixture comprising a compound of formula (I) and fumaric acid of step (i) is stirred at temperature ranging 50° C. to 70° C. In one embodiment, the stirring temperature of step (i) is 55° C. to 65° C. In one embodiment, the stirring temperature of step (i) is 60° C. to 65° C.

In one embodiment, the stirring time of step (i) is 2 to 16 hours. In one embodiment, the stirring time of step (i) is 4 to 10 hours. In one embodiment, the stirring time of step (i) is 4 to 10 hours. In one embodiment, the stirring time of step (i) is 4 hours.

In one embodiment, a step of isolating the compound of formula (I) fumarate from the suspension comprises filtering the compound of formula (I) fumarate obtained from the suspension. In one embodiment, a step of filtering the compound of formula (I) fumarate obtained from the suspension further comprises: washing and drying the compound of formula (I) fumarate obtained from the suspension. In one embodiment, the compound of formula (I) fumarate filtered from the suspension is washed with a solvent. In one embodiment, the compound of formula (I) fumarate is washed with methanol.

In one embodiment, compound of formula (I) fumarate prepared by the method described herein is in substantially pure form. In one embodiment, the purity of the compound of formula (I) fumarate is selected from about 90%, about 91%, about 92%, about 93%, about 94%, about 95%, about 96%, about 97%, about 98%, and about 99%. In one embodiment, the purity of the compound of formula (I) fumarate is selected from about 92%, about 93%, about 94%, about 95%, about 96%, about 97%, and about 98%.

In one embodiment, the compound of formula (I)-fumaric acid cocrystal may have a free base to acid molar ratio of 1 to 1 or 1 to >1, e.g., 1 to 1.3 or 1 to 2, etc.

### Methods for Crystallizing Compound of Formula (I) Fumarate

The solid forms (e.g., crystalline forms) described herein can have certain advantages, for example, they may have desirable properties, such as ease of handling, ease of processing, storage stability, and ease of purification. Moreover, the crystalline forms can be useful for improving the performance characteristics of a pharmaceutical product such as dissolution profile, shelf-life, and bioavailability.

In one embodiment, the present invention provides a method of preparing a crystalline compound of formula (I) fumarate comprising:
a) optionally heating a mixture comprising the compound of formula (I) fumarate and a solvent; and
b) crystallizing the compound of formula (I) fumarate from the mixture.

In one embodiment, the present invention provides a method of preparing a cocrystal of the compound of formula (I) and fumaric acid comprising:
a) optionally heating a mixture comprising the compound of formula (I) fumarate and a solvent; and
b) crystallizing the compound of formula (I) fumarate from the mixture.

In one embodiment of the method of preparing crystalline compound of formula (I) fumarate, the mixture is a solution of the compound of formula (I) fumarate dissolved in the solvent.

In one embodiment of the method of preparing crystalline compound of formula (I) fumarate, the solution comprises the compound of formula (I) fumarate dissolved in a solvent or a mixture of solvents. In some embodiments, the solution comprises a reaction mixture.

In one embodiment, the mixture is a slurry or a suspension. In one embodiment, the slurry or the suspension comprises crude solid material comprising the compound of formula (I).

In one embodiment of the method of preparing crystalline compound of formula (I) fumarate, the solvent is alcohol, ether ketone, ester, chlorinated solvents, nitrile or hydrocarbon; or combination thereof.

In one embodiment of the method of preparing crystalline compound of formula (I) fumarate, the solvent is selected from methanol, ethanol, propanol, pentanol, anisole, isopropanol, butanol, 1,2-dimethoxy ethanol, 2-methoxy ethanol, 2-ethoxy ethanol, ethylene glycol, tetrahydrofuran, diethyl ether, 1,4-dioxane, diisopropyl ether, methyl tert-butyl ether, acetone, methyl isobutyl ketone, dimethylformamide, dimethyl acetamide, dimethyl sulfoxide, ethyl acetate, isopropyl acetate, methyl acetate, n-propyl acetate, n-butyl acetate, isobutyl acetate, chloroform, isobutylene acetate, dichloromethane, dichloroethane, methylcyclohexane, methyl tert-butyl ether (MTBE), acetonitrile, benzene, toluene, xylene, n-hexane, n-heptane, water, and mixtures thereof. In one embodiment of the method of preparing crystalline compound of formula (I) fumarate, the solvent is methanol, ethanol, anisole, isopropanol, butanol, 1,2-dimethoxy ethanol, 2-methoxy ethanol, 2-ethoxy ethanol, ethylene glycol, tetrahydrofuran, diethyl ether, 1,4-dioxane, diisopropyl ether, methyl tert-butyl ether, acetone, methyl isobutyl ketone, dimethylformamide, dimethyl acetamide, dimethyl sulfoxide, ethyl acetate, isopropyl acetate, chloroform, dichloromethane, acetonitrile, benzene, toluene and xylene, or any combination thereof.

In one embodiment of the method of preparing crystalline compound of formula (I) fumarate, the solvent is methanol, ethanol, isopropanol, butanol, 1,2-dimethoxy ethanol, 2-methoxy ethanol, or 2-ethoxy ethanol.

In one embodiment of the method of preparing crystalline compound of formula (I) fumarate, the solvent is methanol, ethanol, isopropanol or butanol. In one embodiment, the solvent is methanol or ethanol.

In one embodiment of the method of preparing crystalline compound of formula (I) fumarate, the solvent is selected from acetonitrile, acetone, anisole, dichloromethane, dichloroethane, ethanol, methanol, methyl acetate, n-propyl acetate, isopropyl acetate, propanol, butanol, pentanol, n-butyl acetate, isobutyl acetate, isobutylene acetate, methylcyclohexane, methyl tert-butyl ether (MTBE), n-hexane, n-heptane, tetrahydrofuran, water, and mixtures thereof.

In one embodiment, the step of crystallizing compound of formula (I) fumarate from the mixture comprises allowing the solvent to evaporate at ambient temperature thereby causing the compound of formula (I) fumarate to precipitate out of solution. In one embodiment, the step of crystallizing compound of formula (I) fumarate from the mixture comprises cooling the mixture to ambient temperature or lower thereby precipitating the compound of formula (I) fumarate cocrystal.

In one embodiment, the present invention provides a method of preparing a crystalline compound of formula (I) fumarate comprising:

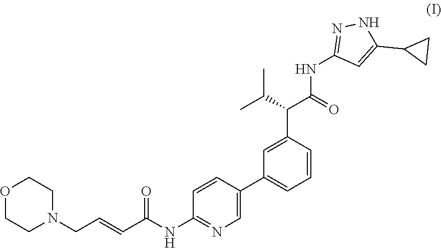 [SMILES: CC(C)[C@H](C(=O)Nc1cc(C2CC2)[nH]n1)c1cccc(-c2ccc(NC(=O)/C=C/CN3CCOCC3)nc2)c1]

comprises:
a) adding an anti-solvent to the mixture comprising a compound of formula (I) fumarate and a solvent; and
b) crystallizing the compound of formula (I) fumarate from the mixture.

In one embodiment, the present invention provides a method of preparing crystalline compound of formula (I) fumarate cocrystal comprising:

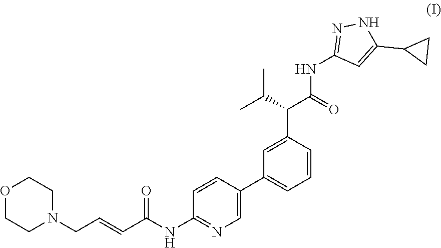 [SMILES: CC(C)[C@H](C(=O)Nc1cc(C2CC2)[nH]n1)c1cccc(-c2ccc(NC(=O)/C=C/CN3CCOCC3)nc2)c1]

comprises:
a) adding an anti-solvent to the mixture comprising a compound of formula (I) fumarate and a solvent; and
b) crystallizing the compound of formula (I) fumarate from the mixture.

In one embodiment, a mixture comprising a compound of formula (I) fumarate and a solvent is heated thereby to form a solution.

In one embodiment of the method of preparing crystalline compound of formula (I) fumarate, the solvent is acetone, n-propyl acetate, acetonitrile, methanol, iso-propyl acetate, iso-butanol, 2-butanol, 1-butanol, n-butyl acetate, 1-pentanol, 1-propanol, chloroform, methyl acetate, isobutyl acetate, iso-butanol or ethanol.

In one embodiment, the mixture comprising the compound of formula (I) fumarate is a solution, and the step of crystallizing the compound of formula (I) fumarate from the mixture comprises bringing the solution to supersaturation thereby causing the compound of formula (I) fumarate to precipitate out of the solution.

In one embodiment, the step of bringing the solution to supersaturation comprises adding an anti-solvent.

In one embodiment, the step of bringing the solution to supersaturation comprises cooling the solution to ambient temperature or lower.

In one embodiment, the step of bringing the solution to supersaturation comprises maintaining a solution temperature above about 20° C.

As used herein, “anti-solvent” means a solvent in which the compound crystals are insoluble, minimally soluble, or partially soluble, i.e., (solubility less than 1 mg/mL). In practice, the addition of an anti-solvent to a solution in which crystals are dissolved reduces the solubility of the crystals in solution, i.e., supersaturation, thereby stimulating precipitation of the subject compound. In one embodiment, the crystals are washed with a combination of anti-solvent and the organic solvent. In one embodiment, the anti-solvent is water, while in other embodiments it is an alkane solvent, such as hexane or pentane, or an aromatic hydrocarbon solvent, such as 1,2-dichloroethane, benzene, toluene, methylcyclohexane or xylene.

In one embodiment, the anti-solvent is dichloromethane, dichloroethane, ethanol, methanol, propanol, butanol, pentanol, isobutyl acetate, isobutylene acetate, methylcyclohexane, n-hexane, n-heptane, tetrahydrofuran, and mixtures thereof.

In one embodiment, the anti-solvent is 1,2-dichloroethane, n-hexane and methylcyclohexane.

In one embodiment, the method of making the crystalline form of compound of formula (I) fumarate are used to remove one or more impurities from a sample of the compound of formula (I)-fumaric acid.

In one embodiment, after crystallization, the compound of formula (I) fumarate is substantially pure. In some embodiments, the crystalline form of the compound of formula (I) fumarate is greater than 90% pure. In some embodiments, the purity of the crystalline form of compound of formula (I) fumarate is selected from greater than 90%, greater than 91%, greater than 92%, greater than 93%, greater than 94%, greater than 95%, greater than 96%, greater than 97%, greater than 98%, and greater than 99%. In some embodiments, the purity of the crystalline form of the compound of formula (I) fumarate is greater than 95%. In some embodiments, the purity of the crystalline form of the compound of formula (I) fumarate is greater than 98%. In some embodiments, the purity of the crystalline form of the compound of formula (I) fumarate is selected from about 90%, about 91%, about 92%, about 93%, about 94%, about 95%, about 96%, about 97%, about 98%, and about 99%.

In one embodiment, crystalline compound of formula (I) fumarate is the crystalline compound of formula (I) fumarate as described herein.

In some embodiments, compound of formula (I) fumarate is substantially isolated.

By “substantially isolated” is meant that the salt, cocrystal or compound is at least partially or substantially separated from the environment in which it was formed or detected. Partial separation can include, for example, a composition enriched in the salts described herein. Substantial separation can include compositions containing at least about 50%, at least about 60%, at least about 70%, at least about 80%, at least about 90%, at least about 95%, at least about 97%, or at least about 99% by weight of the salts described herein, or salt thereof. Methods for isolating compounds and their salts are routine in the art.

In one embodiment, the preparation method further comprises inducing crystallization. The method can also comprise drying the crystals, for example under reduced pressure. In one embodiment, inducing precipitation or crystallization comprises secondary nucleation, wherein nucleation occurs in the presence of seed crystals or interactions with the environment (crystallizer walls, stirring impellers, sonication, etc.).

### Pharmaceutical Compositions

In one embodiment, the invention relates to a pharmaceutical composition comprising a compound of formula (I) fumarate and one or more pharmaceutically acceptable excipients.

In one embodiment, the invention relates to a pharmaceutical composition comprising a compound of formula (I)-fumaric acid cocrystal (1:1) and one or more pharmaceutically acceptable excipients.

In one embodiment, the present invention relates to a pharmaceutical composition comprising a compound of formula (I) fumarate or a cocrystal thereof in combination with at least one other therapeutic agent and a pharmaceutically acceptable carrier or excipient.

### Use of Compound of Formula (I) Fumarate

In one embodiment, the present invention provides a use of pharmaceutical composition comprising a compound of formula (I) fumarate, and at least one pharmaceutically acceptable excipient (such as a pharmaceutically acceptable carrier or diluent). In one embodiment, the present invention provides a use of pharmaceutical composition comprising a compound of formula (I)-fumaric acid cocrystal (1:1) as described herein and at least one pharmaceutically acceptable excipient (such as a pharmaceutically acceptable carrier or diluent). The compound of formula (I)-fumaric acid cocrystal, described in the present invention may be associated with a pharmaceutically acceptable excipient (such as a carrier or a diluent) or be diluted by a carrier or enclosed within a carrier which can be in the form of a capsule, sachet, paper or other container.

In another embodiment, the present invention provides pharmaceutical composition comprising the compound of formula (I) fumarate, for use in treating and/or preventing a disease and/or disorder associated with aberrant activity of selective transcriptional CDKs.

In another embodiment, the present invention provides pharmaceutical composition comprising the compound of formula (I)-fumaric acid cocrystal (1:1), for use in treating a subject suffering from diseases and/or disorder associated with aberrant activity of selective transcriptional CDKs.

In one embodiment, the present invention relates to a pharmaceutical composition comprising a compound of formula (I) fumarate or a cocrystal thereof in combination with at least one other therapeutic agent for use in treating and/or preventing a disease and/or disorder associated with aberrant activity of selective transcriptional CDKs.

In another embodiment, the present invention provides a method of inhibiting selective transcriptional CDKs in a subject, comprising administering to the subject in need thereof a therapeutically effective amount of a compound of formula (I) fumarate, described herein.

In another embodiment, the present invention provides a method of treating diseases and/or disorder mediated by selective transcriptional CDKs in a subject comprising administering to the subject in need thereof a therapeutically effective amount of a compound of formula (I) fumarate, described herein.

In another embodiment, the present invention provides pharmaceutical composition comprising the compound of formula (I) fumarate, described herein, for use in treating a subject suffering from diseases and/or disorder associated with aberrant activity of transcriptional CDK9, CDK12, CDK13 or CDK18.

In another embodiment, the present invention provides pharmaceutical composition comprising the compound of formula (I) fumarate, described herein, for use in treating a subject suffering from diseases and/or disorder associated with aberrant activity of transcriptional CDK7.

In yet another embodiment, the present invention provides a method of treating disorders and/or diseases or condition mediated by selective transcriptional CDKs (CDK9, CDK12, CDK13 or CDK18) in a subject comprising administering a therapeutically effective amount of a compound of formula (I) fumarate, described herein.

In yet another embodiment, the present invention provides a method of treating disorders and/or diseases or condition mediated by transcriptional CDK7 in a subject comprising administering a therapeutically effective amount of a compound of formula (I) fumarate described herein.

In yet another embodiment, the present invention provides a method of inhibiting selective transcriptional CDKs. In another embodiment, the present invention provides a method of inhibiting particularly transcriptional CDK7, CDK9, CDK12, CDK13 or CDK18; more particularly CDK7, in a subject in need thereof by administering to the subject a compound of formula (I) fumarate described herein in the amount effective to cause inhibition of such receptor/kinase.

In another aspect, the present invention relates to methods of inhibiting the activity of a kinase in a biological sample or subject. In one embodiment, the kinase is a selective transcriptional CDK. In another embodiment, the selective transcriptional CDK is CDK7, CDK9, CDK12, CDK13 or CDK18. In yet another embodiment, the selective transcriptional CDK is particularly CDK7.

In one embodiment, the diseases and/or disorder is cancer, an inflammatory disorder, an auto-inflammatory disorder and an infectious disease.

In one embodiment, the inhibition of the activity of the kinase is irreversible. In other embodiments, the inhibition of the activity of the kinase is reversible.

The compound described in the present invention are typically administered in the form of a pharmaceutical composition. Such compositions can be prepared using procedures well known in the pharmaceutical art and comprise at least one compound of the present invention. The pharmaceutical composition of the present invention comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of compounds of the invention, in pure form or in an appropriate pharmaceutical composition, can be carried out using any of the accepted routes of administration of pharmaceutical compositions. The route of administration may be any route which effectively transports the active compound of the present invention to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present invention may be prepared by conventional techniques known in literature.

Suitable doses of the compound of formula (I) fumarate, described herein, for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present invention.

In one embodiment, the compound of formula (I) fumarate as disclosed in the present invention are formulated for pharmaceutical administration.

Yet another embodiment of the present invention provides use of compound of formula (I) fumarate, as disclosed in the present invention in the treatment and prevention of diseases and/or disorder associated with the aberrant activity of selective transcriptional CDKs, particularly the selective transcriptional CDK is CDK7, CDK9, CDK12, CDK13 or CDK18; more particularly CDK7.

Yet another embodiment of the present invention provides use of compound of formula (I) fumarate in treating and/or preventing a disease for which the symptoms thereof are treated, improved, diminished and/or prevented by inhibition of selective transcriptional CDKs, particularly the selective transcriptional CDK is CDK7, CDK9, CDK12, CDK13 or CDK18; more particularly CDK7.

According to yet another embodiment, the selective transcriptional CDK mediated disorder and/or disease or condition is proliferative disease or disorder or condition.

In yet another embodiment, the diseases and/or disorder mediated by selective transcriptional CDKs is selected from, but not limited to the group consisting of a cancer, an inflammatory disorder, an auto-inflammatory disorder or an infectious disease.

In other embodiments, the proliferative disease to be treated or prevented using the compound of formula (I) fumarate will typically be associated with aberrant activity of CDKs, more particularly with CDK7, CDK9, CDK12, CDK13 or 18. Aberrant activity of CDK7, CDK9, CDK12, CDK13 or CDK18 may be an elevated and/or an inappropriate (e.g., abnormal) activity of CDK7, CDK9, CDK12, CDK13 or CDK18. In one embodiment, CDK7, CDK9, CDK12, CDK13 or CDK18 are not overexpressed, and the activity of CDK7, CDK9, CDK12, CDK13 or CDK18 are elevated and/or inappropriate. In certain other embodiments, CDK7, CDK9, CDK12, CDK13 or CDK18 are overexpressed, and the activity of CDK7, CDK9, CDK12, CDK13 or CDK18 are elevated and/or inappropriate.

According to yet another embodiment, the compound of formula (I) fumarate is expected to be useful in the therapy of proliferative diseases such as viral diseases, fungal diseases, neurological/neurodegenerative disorders, autoimmune, inflammation, arthritis, anti-proliferative (e.g., ocular retinopathy), neuronal, alopecia and cardiovascular diseases.

According to yet another embodiment, the compound of formula (I) fumarate is useful in the treatment of a variety of cancers, including but not limited to carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkins lymphoma, non-Hodgkins lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of masenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoctanthoma, xenoderoma pigmentosum, thyroid follicular cancer and Kaposi's sarcoma.

According to yet another embodiment, the subject is a mammal including human.

According to yet another embodiment, the present invention provides compound of formula (I) fumarate, for use as a medicament.

According to yet another embodiment, the invention provides the use of compound of formula (I) fumarate, in the manufacture of a medicament.

According to yet another embodiment, the present invention provides compound of formula (I) fumarate, for use in the treatment of cancer.

According to yet another embodiment, the invention provides the use of compound of formula (I) fumarate in the manufacture of a medicament for the treatment of diseases and/or disorder associated with the aberrant activity of selective transcriptional CDKs.

In yet another embodiment, the invention provides the use of compound of formula (I) fumarate in the manufacture of a medicament for the treatment of cancer.

According to yet another embodiment, the present invention provides compound of formula (I) fumarate for use as a medicament for treating a subject suffering from diseases and/or disorder associated with aberrant activity of selective transcriptional CDKs.

According to yet another embodiment, the present invention comprises administering to the subject in need thereof a therapeutically effective amount of a compound of formula (I) fumarate along with one or more additional chemotherapeutic agents independently selected from anti-proliferative agents, anti-cancer agents, immunosuppressant agents and pain-relieving agents.

The method(s) of treatment of the present invention comprises administering a safe and effective amount of compound of formula (I) fumarate to a patient (particularly a human) in need thereof.

In one embodiment, the present invention provides the compound, which is (S, E)-N-(5-(3-(1-((5-cyclopropyl-1H-pyrazol-3-yl)amino)-3-methyl -1-oxobutan-2-yl)phenyl)pyridin-2-yl)-4-morpholinobut-2-enamide or a pharmaceutically acceptable salt thereof.

In one embodiment, the present invention provides the pharmaceutical composition comprising (S, E)-N-(5-(3-(1-((5-cyclopropyl-1H-pyrazol-3-yl)amino)-3-methyl-1-oxobutan-2-yl) phenyl)pyridin-2-yl)-4-morpholinobut-2-enamide and a pharmaceutically acceptable carrier or excipient. Combination Therapy

In certain embodiments, provided herein are methods for combination therapy of the compound of formula (I) fumarate or a cocrystal thereof with a chemotherapeutic agent, therapeutic antibody, and/or radiation treatment, e.g., to provide a synergistic or additive therapeutic effect for the treatment of diseases, disorders and conditions such as cancer. In certain embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

For example, further therapeutic agent may comprise an alkylating agent, such as chlorambucil, cyclophosphamide, cisplatin; a mitotic inhibitor such as docetaxel or paclitaxel; an antimetabolite such as 5-fluorouracil, cytarabine, methotrexate, or pemetrexed; an anti-tumor antibiotic such as daunorubicin or doxorubicin; a corticosteroid such as prednisone or methylprednisone; a BCL-2 inhibitor such as venetoclax; or immunotherapeutic compound such as nivolumab, pembrolizumab, pidilizumab, avelumab, BMS 936559, or MPDL3280A, or a combination thereof. In one embodiment, the immunotherapeutic compound comprises chimeric antigen receptor T cells (CAR T-cells).

In certain embodiments, the further therapeutic agent is docetaxel. Docetaxel is a type of chemotherapeutic agent known as an antimicrotubule agent. Docetaxel is used for treating a variety of cancers, such as metastatic prostate cancer. Docetaxel treatment is often administered intravenously, and often includes premedication with a corticosteroid such as prednisone. In certain embodiments, the further therapeutic agent is venetoclax which is a BCL-2 inhibitor that can induce apoptosis in cancer cells. Venetoclax is typically administered orally.

In some embodiments, compound of formula (I) fumarate or a cocrystal thereof can be used in combination with one or more chemotherapeutic agents such as, erlotinib, bortezomib, disulfiram, epigallocatechin gallate, salinosporamide A, carfilzomib, 17-AAG (geldanamycin), radicicol, lactate dehydrogenase A (LDH-A), fulvestrant, sunitinib, letrozole, imatinib mesylate, fmasunate, oxaliplatin, 5-FET (5-fluorouracil), leucovorin, Rapamycin, Lapatinib, Lonafamib sorafenib, gefitinib, anti-metabolites such as methotrexate and 5-fluorouracil (5-FU); taxoids, e.g., paclitaxel, ABRAXANE® (Cremophor-free), albumin-engineered nanoparticle formulations of paclitaxel (American Pharmaceutical Partners, Schaumberg, Ill.), and docetaxel/doxetaxel; chloranmbucil; gemcitabine; 6-thioguanine; mercaptopurine; methotrexate; platinum analogs such as cisplatin and carboplatin; retinoids such as retinoic acid; and pharmaceutically acceptable salts, acids and derivatives of any of the above.

In some embodiments, compound of formula (I) fumarate or a cocrystal thereof can be used in combination with one or more additional pharmaceutical agents such as, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies for treatment of diseases, disorders or conditions, such as cancer.

In some embodiments, compound of formula (I) fumarate or a cocrystal thereof as disclosed herein can be used in combination therapy with one or more kinase inhibitors for the treatment of cancer. Exemplary kinase inhibitors include imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilter, larotrectinib, lorlatinib, netarsudil, osimertinib, pemigatinib, pexidartinib, ribociclib, temsirolimus, XL-092, XL-147, XL-765, XL-499, and XL-880.

In some embodiments, compound of formula (I) fumarate or a cocrystal thereof can be used in combination with a HSP90 inhibitor (e.g., XL888), liver X receptor (LXR) modulators, retinoid-related orphan receptor gamma (RORy) modulators, checkpoint inhibitors such as a CK1 inhibitor or aCK1α inhibitor, a Wnt pathway inhibitor (e.g., SST-215), or a mineralocorticoid receptor inhibitor, (e.g., esaxerenone), XL-888 or Poly ADP ribose polymerase (PARP) inhibitors, such as, olaparib, rucaprib niraparib, talzoparib for the treatment of cancer.

In some embodiments, for treatment of cancer, compound of formula (I) fumarate or a cocrystal thereof as disclosed herein can be used in combination with immune check point inhibitors, for example, inhibitors of PD-1 or inhibitors of PD-L1, e.g., an anti-PD-1 monoclonal antibody or an anti-PD-L 1 monoclonal antibody, for example, nivolumab (Opdivo), pembrolizumab (Keytruda, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZM009, AK105, HLX10, and TSR-042, AMP-224, AMP-514, PDR001, durvalumab, pidilizumab (Imfinzi®, CT-011), CK-301, BMS 936559, and MPDL3280A. In some embodiments, the anti-PD-1 monoclonal antibody is nivolumab, pembrolizumab, pidilizumab, PDR001, MGA012, PDR001, AB122, or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab.

In some embodiments, for treatment of cancer, compound of formula (I) fumarate or a cocrystal thereof as disclosed herein can be used in combination with inhibitors of PD-L1. Antibodies that bind to human PD-L1 include atezolizumab, avelumab, durvalumab, tislelizumab, BMS-935559, MEDI4736, FAZ053, KN035, CS1001, CBT-502, A167, STI-A101, CK-301, BGB-A333, MSB-2311, HLX20, KN035, AUNP12, CA-170, BMS-986189 and LY3300054. In some embodiments, the anti-PD-L1 monoclonal antibody is BMS-935559, MEDI4736, MPDL3280A, or MSB0010718C. In some embodiments, the anti-PD-L1 monoclonal antibody is atezolizumab, avelumab, durvalumab.

In some embodiments, for treatment of cancer, compound of formula (I) fumarate or a cocrystal thereof as disclosed herein can be used in combination with CTLA-4 inhibitors, e.g., an anti-CTLA-4 antibody, for example, ipilimumab (Yervoy), tremelimumab and AGEN1884; and phosphatidylserine inhibitors, for example, bavituximab (PGN401); antibodies to cytokines (IL-10, TGF-b, and the like.); other anti-cancer agents such as cemiplimab. In some embodiments, the inhibitor of an immune checkpoint molecule is an inhibitor of PD-L1 and CTLA-4, e.g., an anti-PD-LI/CTLA-4 bispecific antibody or an anti-PD-1/CTLA-4 bispecific antibody. Bispecific antibodies that bind to PD-L1 and CTLA-4 include AK104.

In certain embodiments, the present invention provides a composition comprising compound of formula (I) fumarate or a cocrystal thereof in combination with other therapeutic agents and pharmaceutically acceptable carrier. The compound of formula (I) fumarate or a cocrystal thereof may be administered in combination with one or more other therapeutic agents (preferably one or two, more preferably one): (1) to complement and/or enhance prevention and/or therapeutic efficacy of the preventive and/or therapeutic drug effect of the compound of the present invention, (2) to modulate pharmacodynamics, improve absorption improvement, or reduce dosage reduction of the preventive and/or therapeutic compound of the present invention, and/or (3) to reduce or ameliorate the side effects of the preventive and/or therapeutic compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds).

For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The respective compounds may be administered by the same or different route and the same or different method. In certain embodiments, the other therapeutic compounds can be administered within one hour, 12 hours, 24 hours, 36 hours, 48 hours, 72 hours, or a week prior to or after administration of a compound of formula (I) fumarate or a cocrystal thereof. In certain embodiments, the other therapeutic compounds can be administered within 0.5 hours to 24 hours prior to or after administration of a compound of formula (I) fumarate or a cocrystal thereof. In certain embodiments, the other therapeutic compounds can be administered within 0.5 hours to 72 hours prior to or after administration of a compound of formula (I) fumarate or a cocrystal thereof. In certain embodiments, the other therapeutic compounds can be administered within 2 hours prior to or after administration of a compound of formula (I) fumarate or a cocrystal thereof.

A concomitant medicine comprising the compound of the present invention and other drug may be administered as a combination preparation in which both components are contained in a single formulation, or administered as separate formulations. The administration by separate formulations includes simultaneous administration and or administration of the formulations separated by some time intervals. In the case of the administration with some time intervals, the compound of the present invention can be administered first, followed by another drug or another drug can be administered first, followed by the compound of the present invention, so long as the two compounds are simultaneously active in the patient at least some of the time during the conjoint therapy. The administration method of the respective drugs may be administered by the same or different route and the same or different method.

The dosage of the other drug can be properly selected, based on a dosage that has been clinically used, or may be a reduced dosage that is effective when administered in combination with a compound of the present invention. The compounding ratio of the compound of the present invention and the other drug can be properly selected according to age and weight of a subject to be administered, administration method, administration time, disorder to be treated, symptom and combination thereof. For example, the other drug may be used in an amount of about 0.01 to about 100 parts by mass, based on 1 part by mass of the compound of the present invention. The other drug may be a combination of two or more kind of arbitrary drugs in a proper proportion. The other drug that complements and/or enhances the preventive and/or therapeutic efficacy of the compound of the present invention includes not only those that have already been discovered, but those that will be discovered in future, based on the above mechanism.

In certain embodiments, the present compound of formula (I) fumarate or a cocrystal thereof may be conjointly administered with non-chemical methods of cancer treatment. In certain embodiments, the compound of formula (I) fumarate or a cocrystal thereof may be conjointly administered with non-chemical methods of cancer treatment. In certain embodiments, the compound of formula (I) fumarate or a cocrystal thereof may be conjointly administered with radiation therapy. In certain embodiments, the compound of formula (I) fumarate or a cocrystal thereof may be conjointly administered with surgery, with thermoablation, with focused ultrasound therapy, with cryotherapy, or with any combination of these.

### EXPERIMENTAL

The present invention provides methods for the preparation of compound of formula (I) fumarate according to the procedures of the following examples, using appropriate materials. Those skilled in the art will understand that known variations of the conditions and processes of the following preparative procedures can be used to prepare these compounds. Moreover, by utilizing the procedures described in detail, one of ordinary skill in the art can prepare additional compounds of the present invention.

### Analytical Methods

### X-Ray Powder Diffraction

X-Ray Powder Diffraction patterns were collected on an X'Pert3 PRO MPD diffractometer using CuKa radiation (45 kV, 40 mA).

The details of the data collection are summarized below:

X-Ray Powder Diffraction Parameters

| Instrument | PANalytical |
|---|---|
| Model | X′ Pert3 PRO MPD diffractometer |
| | (Transmission Mode) |
| X-Ray wavelength | Cu, kα, |
| | Kα1 (Å): 1.540598, |
| | Kα2/Kα1 intensity ratio: 0.50 |
| X-Ray tube setting | 45 kV, 40 mA |
| Divergence slit | Fixed 1/2° |
| Scan mode | Continuous |
| Scan range (°2 Theta) | 3°-40° |
| Scan speed [°/min] | 1.2 |
| Step size (°2 Theta) | 0.017 |
| Test Time (s) | 2078 s |
| Temperature | 25° C. (Ambient Temperature) |

### HPLC

Purity analysis was performed on an Agilent HP1100 series system equipped with a diode array detector and using ChemStation software vB.04.03 using the method detailed below.

HPLC Parameters

| Parameter | Value | |
|---|---|---|
| Type of method | Reversed phase with gradient elution | |
| Sample Preparation | Diluent, acetonitrile/H₂O (55:45 v/v) | |
| Column | Sunfire C18, 250 × 4.6 mm, 5 μm | |
| Column Temperature | 45° C. | |
| Injection Volume | 10 μL | |
| Detector Wavelength, Bandwidth | UV at 210 nm | |
| Flow Rate | 1.0 mL/min | |
| Mobile Phase A | 10 mM KH₂PO₄ in H₂O (pH 6.20) | |
| Mobile Phase B | acetonitrile | |
| | Time (min) | % Mobile Phase A |
| Gradient Timetable | 0.0 | 65 |
| | 3.0 | 65 |
| | 30.0 | 40 |
| | 55.0 | 20 |
| | 65.0 | 20 |
| | 66.0 | 65 |
| | 75.0 | 65 |

### Thermogravimetric Analysis and Differential Scanning Calorimetry

Thermogravimetric analysis (TGA) data were collected using a Q5000 TGA from TA Instruments. Differential scanning calorimetry (DSC) was performed using a TA Q2000 DSC from TA Instruments. Method parameters are provided below.

TGA and DSC Parameters

| Parameters | TGA | DSC |
|---|---|---|
| Method | Ramp | Ramp |
| Pan | Platinum, open | Aluminum plate, crimped |
| Temperature | RT-Target Temperature | 25° C.-Target Temperature |
| Ramp rate | 10° C./min | 10° C./min |
| Purge gas | N₂ | N₂ |

The compound of formula (I) was found to be amorphous. In the preparation of compound of formula (I), the final purification required tedious column chromatography procedures which have caused extreme difficulties to obtain desired yield of the compound as impurities were eluting close and multiple purification of impure fractions were required.

With the objective of obtaining the better yield and in order to avoid tedious column purification in the final step of preparation of compound of formula (I), the inventors had performed several trails to crystallize/precipitate the final compound of formula (I)by using various solvent systems however none of the methods was successful. Even salt screening methods by using various acids such as citric acid, succinic acid, tartaric acid etc., with different solvents systems such as ethanol, methanol, IPA, DMSO, DMF, etc., and temperature ranges did not result in any promising yield with better purity. Later it was surprisingly found that compound of formula (I) fumarate was found to be crystalline in nature with superior purity.

Further, compound of formula (I) fumarate has unexpected advantages compared with other salts/cocrystals of compound of formula (I) in various desired properties such as filterability, hygroscopicity, purity and stability. Also, the compound of formula (I) fumarate only showed crystallinity form. Such crystalline material was not obtained with the other acid additions salts/cocrystals.

### Example-1: Preparation of Compound of Formula (I) Fumarate

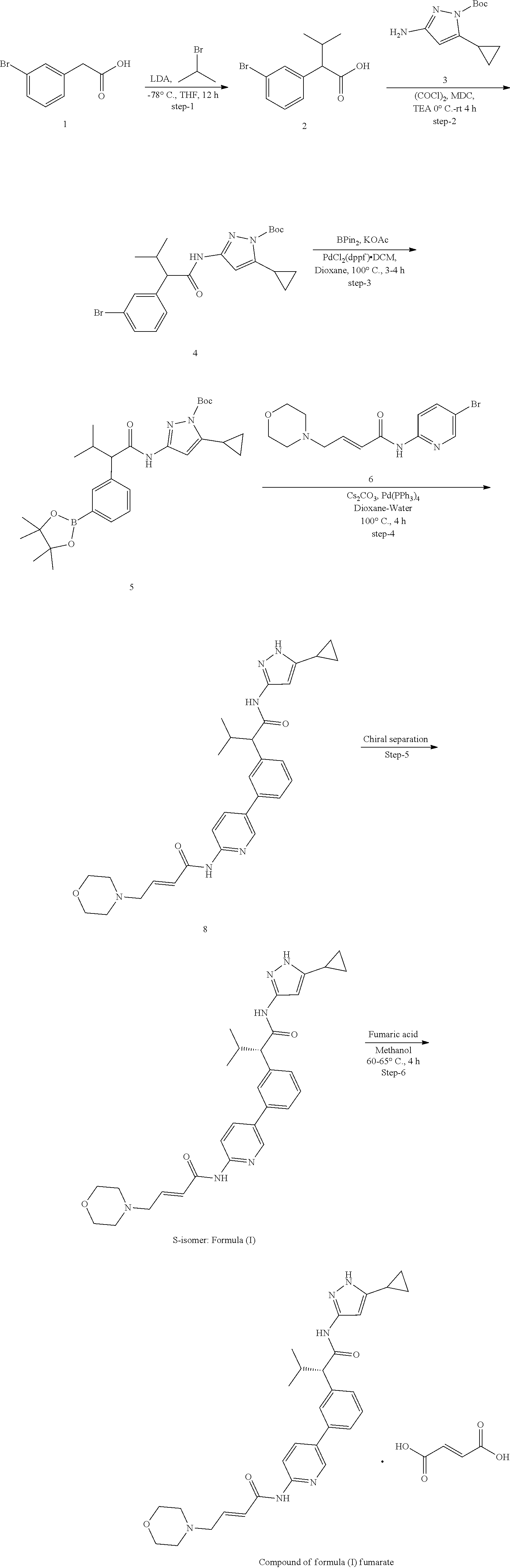

### Step-1: Synthesis of 2-(3-bromophenyl)-3-methylbutanoic Acid

2M LDA (698 mL, 1.38 mol) was added to a solution of 2-(3-bromophenyl) acetic acid (150 g, 0.69 mol) in THF (700 mL) at −78° C. over a period of 30 min. The reaction mass was stirred for 2 h at −78° C. followed by the drop wise addition of isopropyl bromide (255 g, 2.07 mol) over a period of 30 min at −78° C. The reaction mass was stirred at room temperature for overnight. The reaction mass was quenched with 1N HCl (pH 2) and product was extracted to ethyl acetate (500 mL×3). The combined organic layer was washed with water followed by brine, dried and concentrated under reduced pressure to afford the title crude compound which was purified by silica column by eluting with 0-10% ethyl acetate-hexane system to afford the title Compound 2 (150 g, 83% yield). LCMS: m/z=254.80 (M-2H)⁻

### Step-2: Synthesis of tert-butyl 3-(2-(3-bromophenyl)-3-methylbutanamido)-5-cyclopropyl-1H-pyrazole-1-carboxylate

2-(3-Bromophenyl)-3-methylbutanoic acid (70 g, 0.0.27 mol) was dissolved in dry DCM (500 mL) and added oxalyl chloride (68 mL, 0.78 mol) dropwise at 0° C. followed by addition of catalytic amount of DMF (0.8 mL) and maintained the reaction mass at same temperature for 30 min. The reaction mass was allowed to attain room temperature and was stirred for 4 h. The solvent and excess oxalyl chloride was distilled off under vacuum. The obtained residue was re-dissolved in DCM (250 mL) and added slowly the cooled solution of tert-butyl 3-amino-5-cyclopropyl-1H-pyrazole-1-carboxylate (Compound-3, 49 g, 0.218 mol) and TEA (55 mL, 0.546 mol) in THF (250 mL) at 0° C. for 30 min. The reaction was stirred at room temperature for 12 h then the reaction mass was concentrated under reduced pressure and the residue was dissolved in DCM, washed with saturated NaHCO₃ solution and brine. The organic layer was dried over anhydrous sodium sulphate and concentrated under reduced pressure. The obtained crude was purified by silica gel column chromatography by eluting with 15% ethyl acetate-hexane to afford the title Compound 4 (90 g, 71%) LCMS: m/z=363.80 (M-Boc₊₂).

### Step-3: Synthesis of tert-butyl 5-cyclopropyl-3-(3-methyl-2-(3-(4,4,5,5-tetramethyl-1,3,2-dioxaborolan-2-yl)phenyl)butanamido)-1H-pyrazole-1-carboxylate

To a degassed solution of tert-butyl 3-(2-(3-bromophenyl)-3-methylbutanamido)-5-cyclopropyl-1H-pyrazole-1-carboxylate (90 g, 0.193 mol) and 4,4,4′,4′,5,5,5′,5′-octamethyl-2,2′-bi(1,3,2-dioxaborolane) (62 g, 0.251 mol) in 1,4-dioxane (500 mL), was added potassium acetate (37.80 g, 0.386 mol). The reaction mass was stirred for 10 min with degassing at RT and added PdCl₂(dppf).DCM complex (12.5 g, 0.015 mol). The reaction mass was heated for 3-4 h at 100° C. The reaction mixture was cooled to RT and filtered on Celite® bed, and the filtrate was evaporated to get dark brown liquid. The crude material was purified by silica column chromatography by eluting with 20% ethyl acetate in hexane to afford the Compound 5 (90 g, 86%). LCMS: m/z=410 (M-Boc+1)⁺.

### Step-4: Synthesis of (E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide

To a degassed solution of tert-butyl 5-cyclopropyl-3-(3-methyl-2-(3-(4,4,5,5-tetramethyl-1,3,2-dioxaborolan-2-yl)phenyl)butanamido)-1H-pyrazole-1-carboxylate (10 g, 0.019 mol) and (E)-N-(5-bromopyridin-2-yl)-4-morpholinobut-2-enamide (7.7 g, 0.023 mol) in 1,4-dioxane (100 mL) and water (40 mL), Cs₂CO₃ (14.5 g, 0.045 mol) was added. The reaction mass was stirred for 10 min with degassing and added Pd(PPh₃)₄ (1.1 g, 0.00095 mol). The reaction mass was heated for 4 h at 100° C. in a sealed tube. Then, the reaction mass was cooled and diluted with brine solution. The obtained aqueous layer was separated and re-extracted with ethyl acetate. The combined organic layer was evaporated to dryness and crude material was purified by silica column chromatography by eluting with 10-15% methanol in DCM to get pure Compound 8 (4.5 g, 44%). LCMS: m/z=529.15 (M+H)⁺; HPLC: 95.17%, rt: 6.34 min.
Step-5: Chiral Separation

The compound (E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide was separated by using chiral preparative HPLC column (Method: Column: Chiral Pak IA (20 mm×250 mm, 5 micron), Elution: isocratic (50:50), A=ACN, B=MeOH, Flow: 20 mL/min) to afford the pure S and R-Isomers, which are (S, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide and (R, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide, respectively.

S-Isomer: (S, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide (Compound of Formula (I))
¹HNMR (DMSO-d₆, 400 MHz): δ 12.02 (s, 1H), 10.78 (s, 1H), 10.44 (s, 1H), 8.61 (s, 1H), 8.28 (d, 1H), 8.07-8.05 (m, 1H), 7.68 (s, 1H), 7.57 (d, 1H), 7.41-7.37 (m, 2H), 6.81-6.78 (m, 1H), 20 6.49 (d, 1H), 6.13 (s, 1H), 3.61-3.58 (m, 4H), 3.36-3.34 (m, 1H), 3.12 (d, 2H), 2.41-2.32 (m, 5H), 1.82-1.76 (m, 1H), 0.97 (d, 3H), 0.88-0.85 (m, 2H), 0.67 (d, 3H), 0.62-0.59 (m, 2H); LCMS: m/z=529.15 (M+H)⁺; HPLC: 96.72%, rt: 6.39 min; Chiral HPLC: 97.68%, rt: 14.47.
R-Isomer: (R, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide
¹HNMR (DMSO-d₆, 400 MHz): δ 12.02 (s, 1H), 10.78 (s, 1H), 10.44 (s, 1H), 8.61 (s, 1H), 8.28 (d, 1H), 8.07-8.04 (m, 1H), 7.68 (s, 1H), 7.57 (d, 1H), 7.41-7.37 (m, 2H), 6.81-6.78 (m, 1H), 6.50 (d, 1H), 6.14 (s, 1H), 3.61-3.58 (m, 4H), 3.36-3.34 (m, 1H), 3.12 (d, 2H), 2.40-2.39 (m, 5H), 1.82-1.76 (m, 1H), 0.97 (d, 3H), 0.88-0.85 (m, 2H), 0.67 (d, 3H), 0.62-0.60 (m, 2H); LCMS: m/z=529.15 (M+H)⁺; HPLC: 96.24%, rt: 6.39 min; Chiral HPLC: 97.92%, rt: 8.80.

### Step-6: Preparation of Compound of Formula (I) Fumarate

Crude compound of formula (I) was dissolved in methanol (4 vol) and the solution was heated to 60-65° C. and to this was added a solution of fumaric acid (1.1, e.g., fumaric acid was dissolved in methanol, ˜6 vol w.r.t crude compound of formula (I) input). The reaction mixture was stirred at 60-65° C. for 4 h and cooled to room temperature. The obtained suspension was stirred at RT for 16 h. The solid was filtered, washed with 2 vol. of methanol and suck dried for 3 h. The material was further dried at 45-50° C. for 4 h under vacuum to obtain the title compound as a white solid. Yield: 85%.

### Example-2: Polymorph Screening Methods

A. Crystallization from Methanol/Acetone Mixture

The compound of formula (I) fumarate (200 mg) was dissolved in mixture of methanol and acetone (15 mL: 15 mL) in a glass tube at room temperature. The solution was slowly allowed to evaporate at room temperature and crystals were observed after 10 days to give solid form of compound of formula (I) fumarate.
B. Solution Crystallization

The compound of formula (I) fumarate (15 mg) was dissolved in solvents (15 mL) (acetone, ethanol, acetonitrile, isobutylene acetate, isopropyl acetate, n-butyl acetate, n-hexane) in conical flask. The solvent was heated near its boiling point till the formation of clear solution. The solution was allowed to evaporate at room temperature. The crystals were obtained after 10 days to give crystalline form of compound of formula (I) fumarate.
C. Anti-Solvent Crystallization

The compound of formula (I) fumarate (10 mg) was dissolved in solvents (10 mL) (methyl acetate, n-propyl acetate, n-butyl acetate, isopropyl acetate, isobutyl acetate, 1-butanol and acetonitrile) in conical flask near their boiling points. Once clear solution is formed, one of the corresponding anti-solvent (1 mL) (1,2-dichloroethane, n-hexane and methylcyclohexane) was added to the clear solution. The solution was slowly allowed to evaporate at room temperature and crystals were obtained after 10 days to give required solid forms of compound of formula (I) fumarate.
D. Cooling Crystallization

The compound of formula (I) fumarate (10 mL) was dissolved in solvent (10 mL) (methyl acetate, n-propyl acetate, 1-pentanol, methanol and methylcylohexane) in conical flask near the boiling point of the respective solvent till the clear solution is formed. The solution was transferred to ice bath maintained at 2-8° C. and then allowed the crystals to grow. Then the solution was filtered to obtain the solid forms of compound of formula (I) fumarate.
E. Slurry Crystallization

The compound of formula (I) fumarate (20 mg) was added to organic solvent (5 mL) (1,2-dichloroethane, methyl acetate, n-propyl acetate, acetonitrile, isobutanol, 1-butanol, 2-butanol, 1-propanol, n-butyl acetate, isobutyl acetate, isopropyl acetate, n-hexane) in a glass vial at room temperature. The resulting slurry was stirred at 200 rpm for 24 hr. After 24 hours, slurry was filtered to obtain required solid form of compound of formula (I) fumarate.

### Characterization of Compound of Formula (I) Fumarate

The solids, prepared as described herein, were confirmed to be crystalline compound of formula (I) fumarate, by X-ray powder diffraction study (XPRD), Differential Scanning calorimetry (DSC), Thermogravimetric analysis (TGA), Dynamic vapor sorption (DVS) and Single crystal structure studies.
i. Single Crystal X-Ray Crystallographic Analysis

A single crystal of compound of formula (I) fumarate was selected under a polarizing microscope. The selected crystal was used for data collection on a Bruker Kappa Apex2 CCD diffractometer at 100 K. The X-ray generator was operated at 40 kV and 30 mA using Cu Kα (λ=1.5418 A) radiation. Data were collected with w scan width of 1.0 degree. The data reduction followed by Empirical absorption corrections were applied with the various modules within the Apex2 software suite. The structures were solved by direct methods using the SHELXTL package and refined by full-matrix least-squares on F2 from the same. All Non-hydrogen atoms were refined anisotropically, and hydrogen atoms were refined with a riding model. Structure was drawn using Mercury 3.1 and Pymol.

Single crystals of compound of formula (I) fumarate diffracted X-rays better than 1.5 A resolution and diffraction data set with good statistics (Table-2) were collected at room temperature. The structure was solved by direct methods and refined with good geometry. The study successfully determined the complete 3-dimensional structure of the compound of formula (I) and assigned the absolute configuration for the lone chiral center present in the compound. Fumarate makes H-bond contacts with the pyridine ring and its adjacent amide—NH and bridges 2 molecules of the compound with such interactions. Similarly it also forms H-bonds with pyrazole ring N-atom and its adjacent amide—NH group and establishes bridging contacts between two neighbor molecules of the compound.

Structural Information and Refinement Parameters for
compound of formula (I) fumarate

| Crystal data | Structure refinement parameters |
|---|---|
| Empirical formula | C₃₄H₄₀N₆O₇ |
| Molecular weight | 644.73 |
| solvent | Methanol:Acetone (1:1) |
| Temperature/K | 100K |
| Crystal system | Monoclinic |
| Space group | P1211 |
| a/Å | 9.7757 |
| b/Å | 35.3419 |
| c/Å | 9.8641 |
| α/° | 90 |
| β/° | 104.69 |
| γ/° | 90.0 |
| Volume/Å³ | 3296.56 |
| Z | 2 |

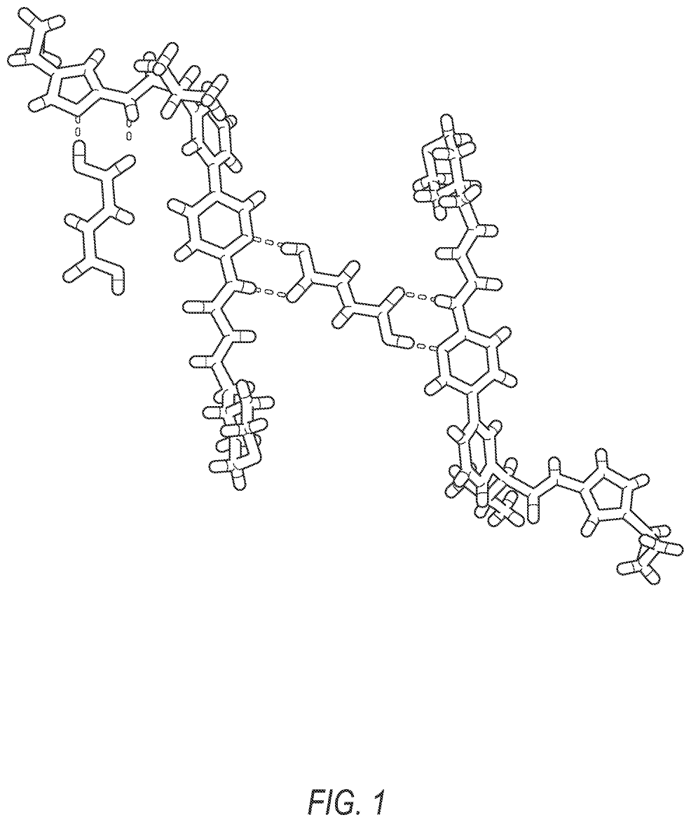

Single crystal X-ray study confirmed the structure of the compound of formula (I) fumarate. This study confirmed the fumaric acid molecule associated with the compound of formula (I) as a cocrystal and also revealed the presence of one molecule of compound of formula (I) and one molecule of fumaric acid in asymmetric unit (molar ratio is 1:1). The measured Hydrogen bond distance between fumarate atoms and N-atoms of compound of formula (I) was between 2.6 to 2.9 A which was typically observed distance for a cocrystal. FIG. 1.
ii. X-Ray Powder Diffraction Study

In one embodiment, compound of formula (I) fumaric acid cocrystal showed characteristic Bragg's peaks at 2-theta values 5.02, 9.99, 10.52, 11.93, 14.98, 18.71, 22.08, 22.51 and 24.03. The X-ray powder diffraction study scan is show in FIG. 2. The peaks of X-ray powder diffraction study are shown in Table. 1.
iii. Differential Scanning Calorimetry

Differential scanning calorimetry (DSC) of compound of formula (I) fumaric acid cocrystal showed an endotherm at 203.9° C. (peak temperature) before melting/decomposition with an onset at 200.2° C. (FIG. 3).
iv. Thermogravimetric Analysis (TGA)

TGA analysis of compound of formula (I) fumaric acid cocrystal was performed in Q5000 TA TGA instrument. Accurately weighed (5-15 mg) sample was loaded in Platinum pan and heated at a rate of 10° C./min over a temperature range of 30 to 300° C. under a nitrogen purge of 50 mL/min. TGA thermogram showed the weight loss initiation after 175° C., indicating absence of adsorbed solvent/moisture. FIG. 4.

### Optical and Polarized Microscopy

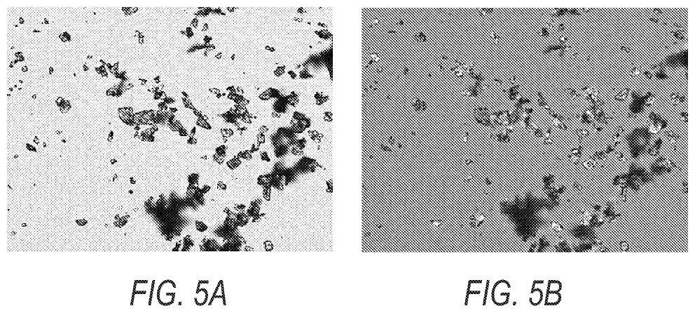

The powder samples were viewed under optical and polarized light microscope (Nikon ECLIPSE, LV100POL equipped with Linkam THMSE 600: TMS94 hot stage) at 100× magnification. Photomicrographs were acquired using Media cybernetics video camera and Qcapture software. In optical microscopy, compound of formula (I)-fumaric acid cocrystal showed irregular shaped/plate shaped crystal habit (FIG. 5A), while polarized light microscopy of compound of formula (I)-fumaric acid cocrystal indicated presence of birefringence pattern thus confirming its crystalline nature (FIG. 5B).

### Example—4: Stability of Compound of Formula (I)—Fumaric Acid Cocrystal

### Variable Temperature X-Ray Diffraction (VT-XRD)

An Anton Paar temperature-humidity chamber (THC) was used to collect in situ XRPD patterns as a function of humidity. The humidity was generated with an RH-200 manufactured by VTI Inc. and carried by a flow of nitrogen gas. The humidity and temperature was monitored by a HygroClip sensor manufactured by Rotronic located next to the specimen inside the THC. XRPD patterns were collected with a PANalytical X'Pert PRO MPD diffractometer in symmetric Bragg-Brentano reflection geometry. Isothermal hold of 60 min given at specified temperature. Heating rate was kept at 10° C./min. Approximately 200 mg sample used for VT-XRD analysis. Cu Kα radiation was produced using a long, fine-focus source operated at 45 kV and 40 mA. The incident beam was conditioned using a nickel filter, a 0.02-rad Soller slit, a 11.6-mm fixed incident beam mask, a fixed 0.76-mm ½° antiscatter slit, and a fixed 0.38-mm ¼° divergence slit. The diffracted beam was conditioned using a 5-mm antiscatter slit, a 0.04-rad Soller slit. The diffraction patterns were collected using a scanning position-sensitive detector (X'Celerator) located 240 mm from the sample. Data were collected and analyzed using Data Collector software v. 5.5.

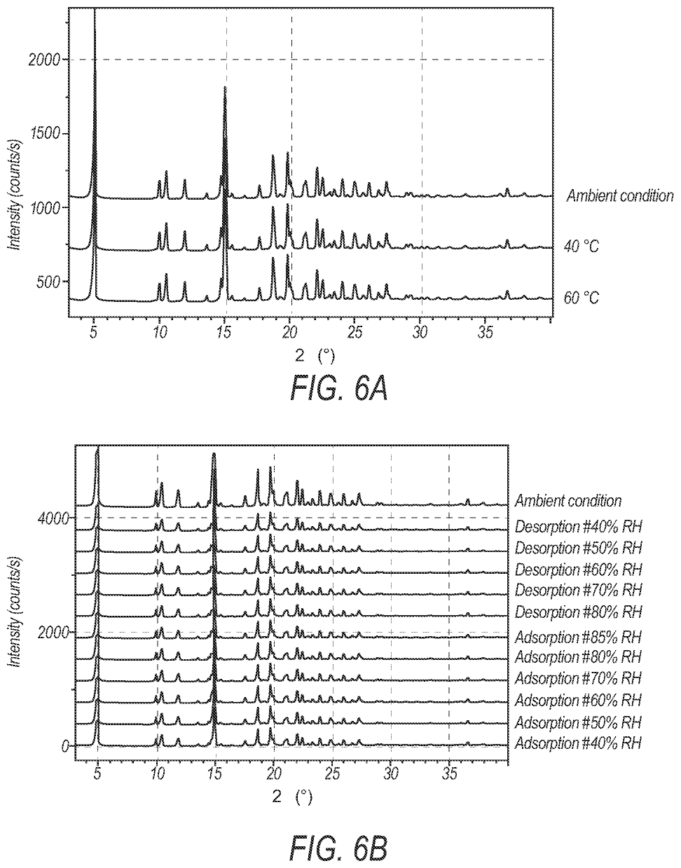

The compound of formula (I) fumarate was exposed to various temperature and was found to be physically stable. VT-XRD analysis confirmed that compound of formula (I)-fumaric acid cocrystal remained stable and no solid form transformation/phase change occurred even at higher temperature conditions. From this observation, the compound of formula (I) fumarate was found to be physically stable at different temperature conditions. See, FIG. 6A.

### Variable Humidity X-ray Diffraction (VH-XRD)

An Anton Paar TTK 450 stage was used to collect in situ XRPD patterns as a function of temperature. The sample was heated with a resistance heater located directly under the sample holder, and the temperature was monitored with a platinum-100 resistance sensor located in the specimen holder. The heater was powered and controlled by an Anton Paar TCU 100 interfaced with Data Collector. Approximately 200 mg sample used for VH-XRD analysis.

The compound of present invention was exposed to various humidity conditions. The sample, i.e., compound of formula (I) fumarate, was exposed from 40% RH to 85% RH followed by desorption cycle from 85% to 40% RH. The samples were equilibrated for 1 hr at each RH level (both sorption and desorption). PXRD pattern collected during this adsorption-desorption cycle did not show any change as a function of relative humidity exposure. From this observation, it can be confirmed that compound of formula (I) fumarate remained physically stable at different relative humidity conditions. See, FIG. 6B.

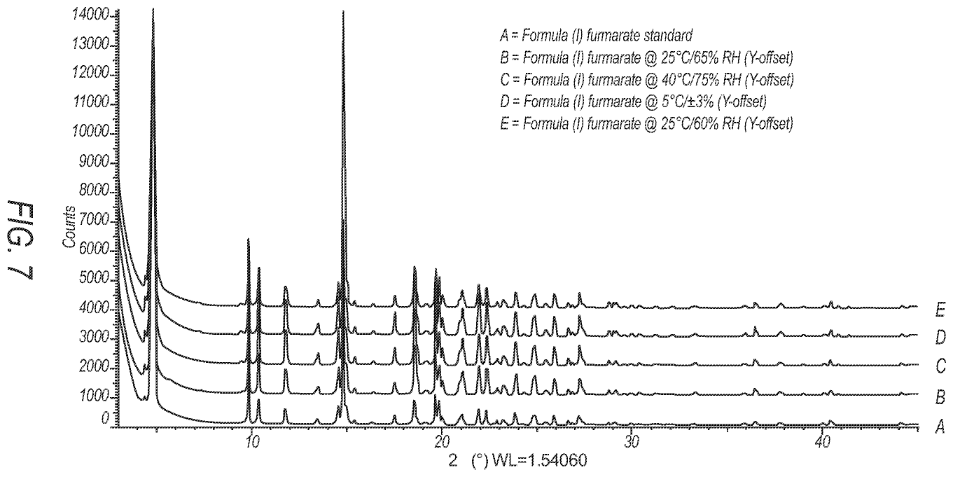

When compound of formula (I) fumarate was further subjected to conditions, 25° C./60% Relative Humidity (RH) and 40° C./75% RH, for 6 months, it was observed that compound of formula (I) fumarate remained unchanged in terms of solid form and particle morphology even after 6 months in the said conditions. See, FIG. 7.

### Dynamic Vapor Sorption (DVS)

The moisture uptake study of compound of present invention was performed with a DVS instrument (Q5000SA, TA instruments, New Castle, Delaware, USA) at 25° C. to evaluate the physical stability of compound of present invention against moisture or tendency to adsorb moisture. The instrument consisted of a microbalance housed inside a temperature-controlled chamber. The humidity was controlled via switching valves, which control the flow of a dry gas (nitrogen) through a humidification stage. Instrument was programmed for moisture sorption from 40 to 90% RH in 10% RH steps at 25±0.1° C. using an equilibrium condition. The equilibrium condition was set to <0.01% total mass change within 10 min and with a maximum dwell time of 60 min followed by desorption from 90% RH to 10% RH steps. The equilibrium condition for desorption was set to <0.01% total mass change within 10 min and with a maximum dwell time of 60 min.

The weight gain/moisture uptake by compound of formula (I) fumarate was increased with increase in the humidity and showed typical “S” shaped curve. About 0.646% w/w weight/moisture was gained by compound of formula (I) fumarate at 90% RH. DVS sorption-desorption isotherm (at 25° C.) of compound of formula (I) fumarate cocrystal is shown in FIG. 8.

### Example—5: Solubility of Compound of Formula (I) Fumarate

Equilibrium solubility of compound of formula (I) free base and compound of formula (I) fumarate were measured in various medium. Excess quantities of compound of formula (I) and compound of formula (I) fumarate were added to glass vials containing different medium. These vials were kept for shaking in shaker water bath at appropriate temperature (25° C. for water and 37° C. for buffers) at 200 rpm. The obtained preparations were filtered and analysed by means of HPLC. The results are given below.

| | | Solubility (mg/ml) ± SD | |
|---|---|---|---|
| Media | pH | Formula (I) free base | Formula (I) fumarate |
| pH 2.8 Acid | 2.8 | 0.065 ± 0.004 | 0.114 ± 0.017 |
| Phthalate buffer USP^{#} | | | |
| pH 4.5 Acetate | 4.5 | 0.07 ± 0.004 | 0.171 ± 0.001 |
| buffer USP^{#} | | | |
| pH 5.8 Phosphate | 5.8 | 0.002 ± 0.0001 | 0.014 ± 0.004 |
| buffer USP^{#} | | | |
| pH 6.8 Phosphate | 6.8 | 0.0005 ± 0.0002 | 0.014 ± 0.009 |
| buffer USP^{#} | | | |
| Purified water** | — | 0.0013 ± 0.0001 | 0.280 ± 0.004 |

^{#}Solubility performed at 37° C. and data at 24 hrs is reported;
**Solubility performed at 25° C. and data at 24 hrs is reported.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A salt which is fumaric acid salt of a compound of formula (I):

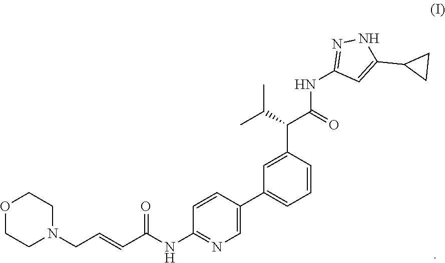 [SMILES: CC(C)[C@H](C(=O)Nc1cc(C2CC2)[nH]n1)c1cccc(-c2ccc(NC(=O)/C=C/CN3CCOCC3)nc2)c1]

2. The salt of claim 1, wherein the salt is crystalline.

3. The salt of claim 2, having Form 1.

4. A cocrystal of a compound of formula (I): 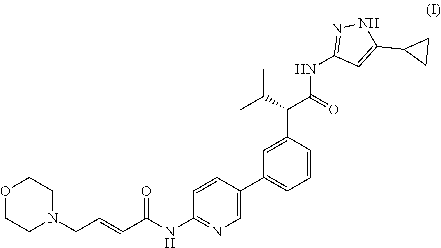 [SMILES: CC(C)[C@H](C(=O)Nc1cc(C2CC2)[nH]n1)c1cccc(-c2ccc(NC(=O)/C=C/CN3CCOCC3)nc2)c1]
and fumaric acid.

5. The cocrystal of claim 4, wherein molar ratio of compound of formula (I) to fumaric acid is 1:1.

6. The cocrystal of claim 4 or 5, characterized by X-ray powder diffraction pattern comprising at least one peak at 2θ angles about 15.0±0.2.

7. The cocrystal of any one of claims 4 to 5, characterized by X-ray powder diffraction pattern comprising at least one peak at 2θ angles selected from about: 5.0±0.2, 10.0±0.2, 10.5±0.2, 15.0±0.2, 18.7±0.2, and 19.8±0.2.

8. The cocrystal of any one of claims 4 to 5, characterized by X-ray powder diffraction pattern comprising at least two peaks at 2θ angles selected from about: 5.0±0.2, 10.0±0.2, 10.5±0.2, 15.0±0.2, 18.7±0.2, and 19.8±0.2.

9. The cocrystal of any one of claims 4 to 5, characterized by X-ray powder diffraction pattern comprising at least three peaks at 2θ angles selected from about: 5.0±0.2, 10.0±0.2, 10.5±0.2, 15.0±0.2, 18.7±0.2, and 19.8±0.2.

10. The cocrystal of any one of claims 4 to 5, characterized by X-ray powder diffraction pattern comprising peaks at 2θ angles about: 5.0±0.2, 10.0±0.2, 10.5±0.2, 15.0±0.2, 18.7±0.2, and 19.8±0.2.

11. The cocrystal of any one of claims 4 to 5, characterized by X-ray powder diffraction pattern comprising at least four peaks at 2θ angles selected from about: 5.0±0.2, 10.0±0.2, 10.5±0.2, 15.0±0.2, 18.7±0.2, 19.8±0.2, 20.0±0.2, 22.0±0.2 and 22.5±0.2.

12. The cocrystal of any one of claims 4 to 5, characterized by X-ray powder diffraction pattern comprising peaks at 2θ angles about: 5.0±0.2, 10.0±0.2, 10.5±0.2, 15.0±0.2, 18.7±0.2, 19.8±0.2, 20.0±0.2, 22.0±0.2 and 22.5±0.2.

13. The cocrystal of any one of claims 4 to 5, characterized by X-ray powder diffraction pattern comprising peaks at 2θ angles about: 5.0±0.2, 10.0±0.2 10.5±0.2, 12.0±0.2, 14.8±0.2, 15.0±0.2, 15.6±0.2, 17.6±0.2, 18.7±0.2, 19.8±0.2, 20.0±0.2, 20.1±0.2, 21.2±0.2, 22.0±0.2, 22.5±0.2, 23.4±0.2, 24.0±0.2, 25.0±0.2, 26.1±0.2, 26.8±0.2, 27.4±0.2, and 36.6±0.2.

14. The cocrystal of any one of claims 4 to 13, characterized by XRD pattern substantially as shown in FIG. 2.

15. The cocrystal of any one of claims 4 to 14, having an endotherm transition at about 195° C. to about 210° C., as measured by differential scanning calorimetry.

16. The cocrystal of any one of claims 4 to 15, having an endotherm transition selected from 195° C. to about 205° C., about 198° C. to about 205° C. and about 199° C. to about 204° C., as measured by differential scanning calorimetry.

17. The cocrystal of claim 15 or 16, wherein the endotherm transition at 203° C.±3° C.

18. The cocrystal of any one of claims 4 to 17, having a thermogravimetric analysis substantially as shown in FIG. 4.

19. The cocrystal of any one of claims 4 to 18, having a dynamic vapor sorption substantially as shown in FIG. 8.

20. A method for preparing a compound of formula (I) fumarate comprising:
a) adding fumaric acid to a mixture comprising the compound of formula (I): 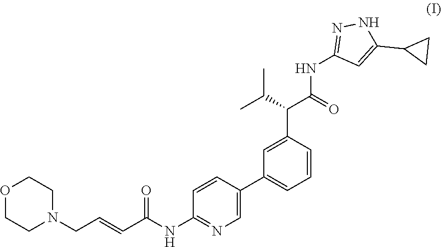 [SMILES: CC(C)[C@H](C(=O)Nc1cc(C2CC2)[nH]n1)c1cccc(-c2ccc(NC(=O)/C=C/CN3CCOCC3)nc2)c1]
and a solvent; and
b) obtaining the compound of formula (I) fumarate from the mixture.

21. The method of claim 20, wherein the mixture comprises a solution of the compound of formula (I).

22. The method of any one of claims 20 to 21, wherein the fumaric acid is dissolved in a solvent.

23. The method of any one of claims 20 to 22, wherein the solvent is methanol, acetonitrile, acetone, anisole, dichloromethane, dichloroethane, ethanol, methyl acetate, n-propyl acetate, isopropyl alcohol, isopropyl acetate, propanol, butanol, pentanol, n-butyl acetate, isobutyl acetate, isobutylene acetate, methylcyclohexane, methyl tert-butyl ether, n-hexane, n-heptane, tetrahydrofuran, or water, or any mixtures thereof.

24. The method of claim 20, wherein obtaining the compound of formula (I) fumarate comprises:
i. stirring the mixture comprising the compound of formula (I) and fumaric acid;
ii. cooling the mixture to ambient temperature thereby forming a suspension;
iii. isolating the compound of formula (I) fumarate from the suspension.

25. The method of claim 24, wherein isolating the compound of formula (I) fumarate comprises filtering the compound of formula (I) fumarate from the mixture.

26. The method of any one of claims 20 to 25, wherein the compound of formula (I) fumarate is in substantially pure form.

27. The method of any one of claims 20 to 26, wherein the purity of the compound of formula (I) fumarate is selected from about 90%, about 91%, about 92%, about 93%, about 94%, about 95%, about 96%, about 97%, about 98%, and about 99%.

28. A method of preparing a crystalline compound of formula (I) fumarate or a cocrystal of the compound of formula (I) and fumaric acid comprising: 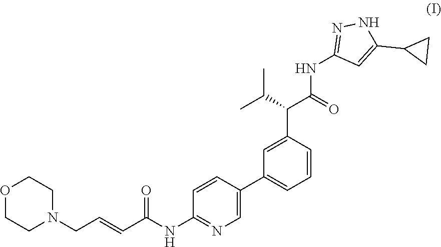 [SMILES: CC(C)[C@H](C(=O)Nc1cc(C2CC2)[nH]n1)c1cccc(-c2ccc(NC(=O)/C=C/CN3CCOCC3)nc2)c1]
a) optionally heating a mixture comprising the compound of formula (I) fumarate and a solvent; and
b) crystallizing the compound of formula (I) fumarate from the mixture.

29. The method of claim 28, wherein mixture is a solution of the compound of formula (I) fumarate dissolved in the solvent.

30. The method of claim 28 or 29, wherein the solvent is selected from methanol, ethanol, anisole, isopropanol, butanol, 1,2-dimethoxy ethanol, 2-methoxy ethanol, 2-ethoxy ethanol, ethylene glycol, tetrahydrofuran, diethyl ether, 1,4-dioxane, diisopropyl ether, methyl tert-butyl ether, acetone, methyl isobutyl ketone, dimethylformamide, dimethyl acetamide, dimethyl sulfoxide, ethyl acetate, isopropyl acetate, chloroform, dichloromethane, acetonitrile, benzene, toluene and xylene, or any combination thereof

31. The method of any one of claims 28 to 30, wherein the step of crystallizing the compound of formula (I) fumarate from the mixture comprises allowing the solvent to evaporate at ambient temperature thereby causing the cocrystal to precipitate out of solution.

32. The method of any one of claims 28 to 30, wherein the step of crystallizing the compound of formula (I) fumarate from the mixture comprises cooling the mixture to ambient temperature or lower thereby precipitating a cocrystal.

33. A method of preparing a crystalline compound of formula (I) fumarate or a cocrystal of the compound of formula (I) and fumaric acid comprising: 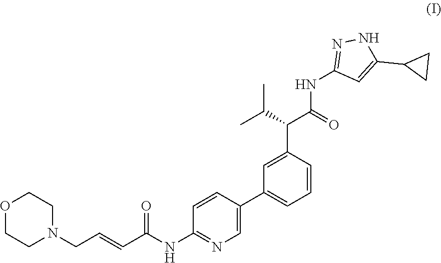 [SMILES: CC(C)[C@H](C(=O)Nc1cc(C2CC2)[nH]n1)c1cccc(-c2ccc(NC(=O)/C=C/CN3CCOCC3)nc2)c1]
a) adding an anti-solvent to a mixture comprising the compound of formula (I) fumarate and a solvent; and
b) crystallizing the compound of formula (I) fumarate from the mixture.

34. The method of claim 33, wherein the mixture is heated to form a solution.

35. The method of claim 34, wherein the solvent is acetone, n-propyl acetate, acetonitrile, methanol, iso-propyl acetate, iso-butanol, 2-butanol, 1-butanol, n-butyl acetate, 1-pentanol, 1-propanol, chloroform, methyl acetate, isobutyl acetate, iso-butanol or ethanol.

36. The method of any one of claims 33 to 35, wherein the mixture comprising the compound of formula (I) fumarate is a solution, and the step of crystallizing the compound of formula (I) fumarate from the mixture comprises bringing the solution to supersaturation thereby causing the compound of formula (I) fumarate to precipitate out of the solution.

37. The method of claim 36, wherein the step of bringing the solution to supersaturation comprises adding an anti-solvent.

38. The method of claim 36, wherein the step of bringing the solution to supersaturation comprises cooling the solution to ambient temperature or lower.

39. The method of claim 36, wherein the step of bringing the solution to supersaturation comprises maintaining a solution temperature above about 20° C.

40. The method of any one of claims 33 to 39, wherein the anti-solvent is dichloromethane, dichloroethane, ethanol, methanol, propanol, butanol, pentanol, isobutyl acetate, isobutylene acetate, methylcyclohexane, n-hexane, n-heptane, tetrahydrofuran, and mixtures thereof

41. The method of claim 40, wherein the anti-solvent is 1,2-dichloroethane, n-hexane and methylcyclohexane.

42. The method of any one of claims 28 to 41, wherein the mixture comprising the compound of formula (I) fumarate is a slurry.

43. The method of any one of claims 28 to 42, further comprising isolating the crystalline compound of formula (I) fumarate.

44. The method of claim 43, wherein isolating the crystalline the compound of formula (I) fumarate comprises filtering the crystalline the compound of formula (I) fumarate from the mixture.

45. The method of any one of claims 28 to 44, wherein the compound of formula (I) fumarate is in a substantially pure form.

46. The method of claim 45, wherein the purity of the crystalline compound of formula (I) fumarate is selected from about 90%, about 91%, about 92%, about 93%, about 94%, about 95%, about 96%, about 97%, about 98%, and about 99%.

47. The method of any one of claims 28 to 46, wherein the crystalline compound of formula (I) fumarate is the crystalline the compound of formula (I) fumarate of any one of claims 1 to 7.

48. A pharmaceutical composition comprising the compound of formula (I) fumarate or a compound of formula (I) fumarate cocrystal according to any one of claims 1 to 19 and one or more pharmaceutically acceptable excipients.

49. A method of treating diseases and/or disorder mediated by selective transcriptional CDKs in a subject comprising administering to the subject in need thereof a therapeutically effective amount of a compound of formula (I) fumarate according to anyone of claims 1 to 19.

50. The method of claim 49, wherein the selective transcriptional CDKs are CDK7, CDK9, CDK12, CDK13 or CDK18.

51. The method of claim 50, wherein the diseases and/or disorders mediated by selective transcriptional CDKs is selected from the group consisting of a cancer, an inflammatory disorder, an auto-inflammatory disorder and an infectious disease.

52. The method of claim 51, wherein the cancer is cancer is selected from the group consisting of a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkins lymphoma, non-Hodgkins lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of masenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoctanthoma, xenoderoma pigmentosum, thyroid follicular cancer and Kaposi's sarcoma.

53. A compound, which is (S, E)-N-(5-(3-(1-((5-cyclopropyl-1H-pyrazol-3-yl) amino)-3-methyl-1-oxobutan-2-yl)phenyl)pyridin-2-yl)-4-morpholinobut-2-enamide or a pharmaceutically acceptable salt thereof.

54. A pharmaceutical composition comprising (S, E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide and a pharmaceutically acceptable carrier or excipient.
